SEQUENCE LISTING (6 sequences)

SEQ ID NO: 1 - DNA, 6423 bp, Artificial Sequence
  Synthetic construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaccatgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgacggaagtggagctaccaattttagtctgctgaagcaggccggcgacgtggaggagaaccccggccccatgtctcgctccgtggccttagctgtgctcgcgctactctctctttctggcctggaggctgtgatggcgccgagaacgctaatactaggatgcggtgctagcggtggtggtggtagcggaggtggaggcagcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggcggtggtggttccggtggaggcggttccggaggtggtggatccggtggtggaggtagtggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctgctacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgggaagcggagagggcagaggaagtctgctaacatgcggtgacgtcgaggagaatcctggacctatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgactggaagcggacagtgtactaattatgctctcttgaaattggctggagatgttgagagcaaccctggacctaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcat

SEQ ID NO: 2 - DNA, 6348 bp, Artificial Sequence
  synthetic construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggcggccgccatggaaccccctggaaggagagagtgccctttcccttcctggagatttcccggactgctgctggctgccatggtcctgctgctgtacagcttcagcgatgcttgtgaggagcctcccacattcgaggccatggaactcattggcaagcctaagccctactacgaaatcggcgagagggtcgattacaagtgtaagaagggctatttctacatcccccccctggccacacacaccatctgcgatagaaaccacacatggctccctgtgtccgacgatgcttgctatagggagacctgcccttacatcagggaccctctgaatggacaggccgtgcctgctaatggcacctacgaattcggctatcaaatgcatttcatctgcaatgagggctactacctgatcggcgaagagattctctactgtgagctcaagggaagcgtcgctatctggagcggaaagcctcccatctgcgagaaagtgctgtgcacccccccccctaaaatcaaaaatggcaagcacaccttcagcgaggtggaagtgttcgaatatctggatgccgtgacctacagctgtgatcctgctcctggacccgatccctttagcctgattggcgagagcacaatttactgcggagacaactccgtgtggagcagagccgcccccgagtgtaaggtcgtgaaatgtaggttccctgtcgtcgagaatggcaaacagatcagcggcttcggcaaaaagttctactataaagctaccgtgatgtttgagtgcgacaagggattctacctcgatggcagcgatacaatcgtgtgcgatagcaactccacatgggacccccctgtccccaaatgcctgaaagtgagcaccagctccacaaccaagtcccccgcctcctccgcctccggccccagacctacctataagccccctgtgtccaactaccccggctaccccaaacccgaggaaggcattctggactccctggatgtgtgggtgattgccgtcatcgtcattgctattgtggtcggagtcgctgtgatctgtgtcgtgccctatagatacctgcagagaaggaaaaagaagggcacctacctgaccgacgagacccacagagaagtgaagtttaccagcctgggatccggagccaccaactttagcctgctcaaacaagccggcgacgtggaagagaaccccggacctatgtccagatccgtggccctggctgtgctggctctgctgtccctgagcggcctcgaggccaaaggaccccccgctgctctgacactgggctgtggcgcttccggaggcggcggcagcggaggcggaggcagcattcagaggacccctaagatccaggtgtactccaggcatcctgccgagaacggcaaatccaatttcctcaattgctacgtgtccggcttccatccctccgacatcgaagtggacctcctcaagaacggagagagaatcgaaaaggtggagcactccgacctgtccttctccaaagactggtccttctacctgctgtattacacagagtttacccctaccgagaaggacgagtacgcctgtagagtgaatcacgtgacactgtcccagcccaagattgtgaaatgggacagggacatgggcggcggaggaagcggaggcggcggaagcggcggcggcggcagcggcggcggcggctccggctcccacagcatgaggtacttttccgccgccgtgtccaggcccggcaggggcgaacctagatttatcgctatgggctacgtcgacgacacccagtttgtgaggttcgacagcgactccgcctgccccagaatggaacctagagccccttgggtcgagcaggaaggccccgagtactgggaggaggagaccaggaataccaaggcccacgctcagaccgacaggatgaatctgcagaccctgaggggatgctacaaccaaagcgaggccagctcccacaccctgcagtggatgatcggctgtgatctgggatccgacggcaggctcctgagaggctatgagcagtacgcctacgatggcaaggattacctggctctcaacgaggacctcagatcctggacagccgccgatacagccgctcagatctccaagaggaagtgcgaggctgccaatgtcgctgagcagaggagggcctacctggaaggaacctgcgtcgaatggctgcacaggtacctggaaaacggcaaggaaatgctccagagggccgaccctcctaagacacacgtcacacaccaccccgtgttcgattatgaggccacactcaggtgctgggctctcggcttttatcccgccgagattatcctgacctggcagagagatggcgaggatcagacccaggacgtcgagctggtggagacaaggcctgccggcgacggaacatttcagaaatgggctgctgtggtggtgccctccggcgaggaacagaggtacacatgccacgtgcagcatgaaggactccccgagcccctgatgctgagatggaagcagtcctccctgcctacaatccccatcatgggcatcgtggctggcctcgtcgtcctcgctgctgtggtgaccggcgctgccgtcgccgccgtgctgtggagaaagaagtcctccgatggaagcggcgagggaaggggaagcctgctgacatgcggcgatgtggaggagaaccctggacctatgtggcctctcgtcgctgccctgctgctgggatccgcctgctgtggatccgcccagctcctgtttaataaaacaaagagcgtggagtttaccttttgtaacgataccgtggtgatcccctgcttcgtcaccaacatggaagcccagaataccaccgaagtgtatgtcaagtggaagttcaaaggcagggacatttacaccttcgatggcgctctgaacaaaagcaccgtgcccacagatttcagctccgccaagatcgaagtgtcccagctgctgaagggcgatgctagcctgaaaatggacaaatccgacgccgtgagccacaccggcaactatacctgcgaggtcacagaactgacaagggaaggcgagacaatcatcgagctgaaatacagagtggtgtcctggttctcccctaacgagaacattctgatcgtgattttccctatctttgccattctgctgttctggggccagtttggcatcaagaccctgaagtatagatccggcggcatggatgagaaaaccattgccctcctggtggctggcctggtcattaccgtgatcgtgatcgtgggcgctatcctcttcgtccctggcgagtacagcctgaaaaacgccaccggactgggactgatcgtgacctccacaggaatcctgattctgctgcactattacgtgtttagcaccgctatcggcctgacctcctttgtgatcgccatcctggtgatccaggtcatcgcttatatcctggccgtcgtcggactgagcctctgtatcgccgcctgtatccccatgcatggccccctcctcatcagcggactctccattctggccctggcccaactcctgggcctcgtgtacatgaagttcgtggcctccaaccaaaaaacaatccagcctcccagaaaggccgtcgaggagcccctgaacgcttttaaggagagcaaaggcatgatgaacgatgagggatccggacagtgtaccaactatgccctcctgaaactggctggcgacgtggagagcaatcctggacctatgggcatccagggcggctccgtcctgttcggcctcctcctcgtcctggccgtcttttgccattccggccattccctccagtgctacaactgtcctaaccccaccgccgactgtaagacagctgtgaactgtagcagcgacttcgatgcttgcctgatcacaaaggctggactgcaggtgtacaataagtgctggaagttcgagcactgcaacttcaatgacgtgacaaccaggctgagggaaaacgagctgacctactactgctgcaaaaaggacctgtgcaatttcaatgagcagctggagaatggcggcacatccctgagcgagaaaaccgtgctgctgctggtgacccctttcctggccgccgcttggtccctgcaccccggctccggagtgaagcagaccctcaacttcgatctcctcaaactggccggagacgtcgaaagcaatcctggacctatgaccgagtacaagcctacagtgagactggccaccagggatgatgtgcccagagccgtcagaaccctggctgccgctttcgctgattaccccgctacaaggcataccgtggaccctgatagacacattgagagggtcacagagctgcaggagctgttcctgacaagggtgggcctggatatcggaaaggtctgggtggccgatgacggagctgctgtcgccgtctggacaacacctgagagcgtggaggccggagccgtgtttgccgaaattggacctaggatggctgagctgtccggctccaggctcgctgcccagcagcagatggagggcctgctcgcccctcatagacctaaagagcccgcctggttcctggctaccgtgggagtgtcccccgatcaccagggaaaaggcctgggatccgccgtcgtgctccctggagtcgaagctgccgaaagggctggcgtccccgcttttctcgaaacatccgcccccagaaacctgcccttctacgaaaggctgggcttcaccgtcacagctgacgtcgagtgccctaaggatagagctacctggtgcatgacaaggaagcctggagcttgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 3 - DNA, 7215 bp, Artificial Sequence
  synthetic construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggcggccgccatgctggagggcgtgcaggtcgagacaatcagccctggagacggaaggaccttccccaaaagaggccaaacatgcgtggtccattacaccggaatgctcgaggacggcaaaaaggtggacagcagcagggataggaacaaacccttcaaattcatgctgggaaaacaagaggtgatcagaggatgggaggaaggagtggcccaaatgtccgtgggacagagggccaagctgacaatctcccccgattacgcctacggcgctacaggccatcctggaatcatccccccccacgccacactggtgttcgatgtggaactcctcaagctggagtccggaggaggctccggagtggacggatttggcgacgtgggagctctggaaagcctcagaggaaacgccgacctcgcctatattctgtccatggagccctgtggccattgcctcatcattaataacgtgaacttctgcagggaaagcggactgaggacaagaaccggcagcaacattgactgtgagaaactgagaaggagattcagctccctgcatttcatggtggaggtgaagggcgatctgacagccaagaaaatggtcctggccctgctcgagctggccagacaggaccacggagctctggactgctgtgtggtcgtcatcctcagccacggatgccaagctagccatctgcaattccccggcgctgtgtatggaaccgatggctgtcccgtgtccgtggaaaaaatcgtcaatatctttaacggcacaagctgcccctccctcggaggcaaacctaagctgttctttattcaagcttgcggcggcgagcaaaaggaccatggctttgaagtggcttccacctcccccgaagacgagagccccggatccaatcctgagcccgatgctacccccttccaggagggcctcaggaccttcgaccagctcgatgccatcagcagcctgcctaccccttccgacatcttcgtgtcctacagcacctttcccggattcgtgagctggagggatcccaagagcggaagctggtatgtggagacactggacgatatttttgagcagtgggcccacagcgaggatctgcaatccctgctgctgagagtcgccaacgccgtgagcgtgaaaggaatctacaagcagatgcccggatgcttcaacttcctgaggaagaagctgttcttcaagaccagcgtggactatccctatgatgtgcccgactacgccctcgatggctccggagccaccaacttcagcctgctcaagcaagccggcgacgtggaggagaaccctggacctatggaagtgtcctccagatccagcgagcctctggatcccgtgtggctcctggtcgccttcggaagaggaggcgtcaagctggaggtcctgctgctgtttctgctccccttcacactgggccactgtcctgcccctagccaactgccctccgccaagcctatcaacctcaccgatgagtccatgtttcccattggcacctacctcctgtatgagtgcctccctggatatattaagagacagttctccatcacatgcaagcaggattccacctggaccagcgccgaagacaagtgcatcaggaagcagtgcaaaaccccttccgatcccgaaaatggactcgtgcacgtgcacacaggaatccagttcggctccagaattaattatacatgtaatcagggctacagactgatcggcagctccagcgctgtctgtgtgattaccgatcagagcgtggattgggacaccgaggcccctatctgtgagtggatcccttgcgagattccccctggcattcctaacggcgacttctttagctccaccagagaggatttccactacggcatggtcgtcacctacaggtgcaacacagacgccaggggaaaagccctgttcaacctggtcggcgagccttccctctattgcacatccaatgacggcgaaatcggcgtctggagcggacctcccccccaatgcatcgaactgaacaagtgcacaccccccccttatgtggagaatgccgtcatgctcagcgaaaataggtccctgttttccctgagggacatcgtggagttcaggtgtcaccctggcttcatcatgaagggagccagcagcgtgcattgccagagcctcaataagtgggagcccgagctgccctcctgcttcaagggcgtcatttgcagactgccccaggagatgagcggcttccaaaagggcctcggcatgaagaaagagtactactatggcgaaaatgtcaccctggaatgcgaggatggctatacactcgagggcagctcccagagccagtgccagagcgatggcagctggaaccctctcctggctaagtgcgtgtccaggagcatcagcggcctgattgtgggcatctttattggaattattgtcttcatcctcgtgatcattgtgtttatctggatgattctgaagtacaaaaaaagaaacaccacagatgaaaagtataaagaggtgggcattcatctcaactacaaggaggatagctgcgtgagactgcagtccctgctcacatcccaagaaaatagcagcaccacatcccccgccagaaattccctcacccaagaggtgtccggaagcggcgagggaaggggaagcctcctgacatgcggcgatgtggaagaaaatcccggccctatgagggcccagaggggcctgatcctgctgctcctcctgctggccgtgttctgcagcacagccgtgtccctcacctgctaccattgcttccagcctgtcgtgtccagctgcaacatgaatagcacatgctcccctgaccaggattcctgcctgtacgctgtcgctggaatgcaggtgtaccagaggtgctggaagcaaagcgactgtcatggcgagatcatcatggatcagctggaggagaccaagctgaagttcagatgttgtcagtttaatctctgtaacaagagcgatggaagcctcggaaaaacccctctcctcggaacaagcgtgctcgtcgccattctgaatctctgctttctgtcccatctgggatccggccaatgcaccaactatgccctcctcaaactcgccggagatgtggaatccaaccccggccctatggctaggtccgtcaccctggtcttcctggtgctggtgagcctgaccggcctgtatgctgctatggctcccaggacactcctgctgggctgtggagcctccggaggaggcggcagcggaggcggcggctccattcagaagacacctcaaatccaggtgtactccagacacccccccgagaacggaaagcctaatattctcaattgttacgtgacccagtttcaccccccccatattgagatccagatgctcaaaaacggcaagaaaattcctaaggtggagatgagcgacatgagctttagcaaggactggagcttctacattctcgcccacacagaatttacacccaccgagaccgacacctatgcttgcagggtgaaacatgccagcatggctgagcctaagacagtgtactgggatagggacatgggaggaggcggatccggcggcggcggaagcggcggaggcggctccggcggcggaggaagcatggccctgggaagactcctcctcctgctggctgccgctctgaccctgaccaaaaccggcgccggcagccactccctcagatactttgaaacctccgtgagcagacctggcttcggcaagcctagattcatcagcgtcggctacgtggacgatacacaattcgtcaggttcgactccgacgccaagaaccccagatacgaacccagggccccttggatggagcaggaaggccctgaatattgggagagaaataccagaagagtgaagggatccgagaagaggtttcaggaaagcctgagcaccctcctgagctgctataaccagagcaagggcggcatccacaccttccagaaactgtccggatgcgatctgggctccgacggaagactccagagcggatacctccagttcgcctacgatggcctggactacattgccctcaatgaggacctcgagacctggaccgccgctgatgtggccgcccaagaaaccaggaggaagtgggagcaggctggagccgctgaaaagcacaggacctacctcgagggcaaatgcctgatgtggctgcatagatatctcgaactgggcaaggagatgctgctgaggaccgatcctcctaaggcccacgtcacacatcatcccaggagccagggcgatgtgaccctgagatgttgggctctgggcttctatcctgccgatattaccctcacctggcagctcaacggagaggagctcacccaggacatggagctggtcgaaacaagacccgccggcgacggaacatttcagaaatgggcctccgtggtggtccccctgggcaaagagcagaattacacctgccacgtgtaccacgagggactgcctgagcccctgacactgaggtgggaaccccccccctacacagtctccaacatggtgatcatcgccgtcctcgtggtgctcggcgctgtgattgtgattggcgccgtggtgatcatcggcgtcatggtgagcttcgtgatgaagagaaggaggaacaaaggaggccagggagaagattgtgctctggcccctagcagagattccgcccagagctccgacattagcctcctggactgtaaagccggcagcggagtgaagcagaccctcaacttcgacctgctgaagctggccggagacgtggagagcaatcctggacccatgatcgagcaggacggactccatgctggaagccctgccgcttgggtcgagaggctctttggatacgactgggctcagcagacaatcggctgcagcgatgccgccgtgtttagactcagcgcccagggcagacctgtgctgtttgtgaagaccgacctgagcggagctctcaatgagctccaagatgaagccgccagactgagctggctcgccaccacaggcgtgccttgtgctgccgtgctggacgtcgtcacagaggccggaagggattggctgctcctgggcgaagtccccggacaggatctgctgagctcccatctggcccctgccgaaaaggtctccattatggccgatgccatgaggaggctgcacaccctggatcccgctacctgtccctttgaccatcaggccaagcacaggattgagagggccagaacaagaatggaggccggcctggtcgaccaggatgatctggacgaggaacaccagggcctcgctcctgccgagctctttgccagactcaaggccaggatgcctgacggcgaagatctggtggtgacccatggagatgcctgtctccccaacatcatggtggaaaacggcaggttctccggattcattgactgcggcaggctcggcgtggccgacaggtatcaggacatcgccctggctaccagagatatcgctgaggagctgggaggagagtgggccgatagatttctcgtgctctatggcattgccgctcccgacagccagagaattgccttctacagactgctcgatgagtttttttgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcat

SEQ ID NO: 4 - DNA, 7211 bp, Artificial Sequence
  synthetic construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggcggccgcaccatggccagctatccttgtcaccagcatgcctccgcttttgaccaggccgctagatccagaggccattccaacaggaggacagccctcagacccaggaggcagcaagaggccacagaggtcaggctggagcagaaaatgcccaccctcctgagggtgtacattgatggccctcacggcatgggaaagaccacaacaacccaactgctggtcgccctgggctccagggatgatattgtgtacgtgcccgaacccatgacctactggagggtgctgggcgcctccgagaccatcgctaatatctacaccacccaacacaggctcgatcagggcgagatttccgctggcgatgctgctgtggtcatgacctccgcccagatcaccatgggcatgccctacgccgtgacagatgccgtcctggctcctcatatcggaggagaagccggcagctcccatgctcctcctcccgccctcacactgatctttgacagacaccccatcgccgccctcctgtgttaccctgccgctagatacctgatgggaagcatgacaccccaggccgtgctcgctttcgtggctctgattccccctaccctgcccggcacaaacatcgtcctgggcgctctgcccgaagataggcacatcgataggctcgccaaaaggcaaaggcccggagaaagactggacctcgctatgctcgctgctattaggagagtgtacggcctcctcgccaacaccgtgagatatctccagggcggcggcagctggagagaggattggggccagctgtccggcgctgccgtgcctcctcagggagccgaacctcaaagcaatgctggccctagaccccacatcggcgacaccctcttcaccctgtttagggcccccgaactgctcgcccccaacggcgacctctacaacgtctttgcctgggccctggacgtgctggccaaaaggctgaggcctatgcacgtgttcatcctggactatgaccagtcccccgccggctgcagggatgccctcctccagctgaccagcggaatggtgcagacccacgtgacaacccccggcagcatccccaccatttgcgacctggccaggacattcgccagagagatgggcgaagctaatggatccggagccaccaacttcagcctgctgaagcaagccggcgacgtggaggaaaaccccggccccatggccaggtccgtgacactcgtcttcctggtgctggtgtccctgaccggactgtatgctatggtcatgagcttcgtgaatctgggatgcggcgccagcggaggcggaggctccggcggcggcggcagcatccaaaagacacctcagatccaggtgtattccaggcaccctcctgaaaatggcaaacctaatattctcaactgttacgtcacacagtttcaccccccccacattgaaatccagatgctgaagaacggcaagaaaatccccaaggtcgaaatgagcgacatgagcttcagcaaggactggtccttttacatcctggctcacaccgagtttacccctaccgagaccgatacctacgcctgcagagtgaagcacgccagcatggccgagcctaagacagtctactgggacagagacatgggaggcggcggcagcggcggcggcggcagcggaggaggaggctccggaggaggcggcagcggaccccactccctgaggtactttgtcacagccgtcagcagacccggactcggagagcctaggtatatggaagtgggatacgtggacgacaccgagttcgtgaggtttgactccgacgctgagaaccctagatacgaacccagggccaggtggatggagcaagagggccctgagtattgggaaagggaaacccagaaggccaaaggcaacgaacagagcttcagggtcgacctcagaacactgctgggctgctataaccagagcaagggaggcagccataccatccaggtgatcagcggctgcgaggtcggatccgacggcagactgctgaggggatatcagcagtacgcttacgacggctgcgattatattgccctgaacgaggatctgaaaacctggaccgctgctgatatggctgccctgatcaccaagcataagtgggaacaggctggcgaggctgagaggctgagagcttacctcgaaggcacatgtgtcgagtggctgaggaggtacctcaagaacggcaatgccacactcctgagaaccgatagccctaaggctcacgtcacacatcatagcaggcccgaggacaaggtcaccctgagatgttgggccctgggattttaccccgccgatatcaccctgacctggcaactgaacggagaggagctgatccaggatatggaactggtggaaaccagacctgctggcgacggcaccttccaaaagtgggccagcgtggtcgtccctctgggaaaagagcaatactatacctgccacgtctaccatcagggcctgcctgagcctctcaccctgagatgggagcctccccccagcaccgtcagcaatatggccaccgtggctgtgctcgtggtgctgggagccgccattgtgacaggcgccgtggtggccttcgtcatgaagatgaggaggaggaacaccggaggcaaaggcggagactatgccctcgctcccggcagccagacatccgacctcagcctgcccgactgtaaggctggctccggcgaaggcagaggaagcctcctgacctgtggagatgtcgaggaaaatcccggccccatgtggcccctggctgctgccctcctgctgggaagctgttgctgtggctccgctcagctgctgtttagcaacgtcaactccatcgagtttacaagctgtaacgagaccgtggtcatcccctgtatcgtgaggaacgtggaggcccagtccaccgaagagatgttcgtcaagtggaaactgaacaaaagctatatcttcatttacgacggaaataaaaactccaccaccacagaccagaactttaccagcgccaaaatctccgtctccgatctcatcaatggcatcgccagcctgaagatggacaagagggatgctatggtcggcaattacacctgcgaggtgacagaactcagcagagagggcaagaccgtgatcgagctcaaaaataggaccgcctttaatacagaccaaggctccgcttgcagctatgaagaggaaaaaggaggatgcaagctcgtcagctggttttcccccaatgagaagattctcattgtgatcttccccatcctggccattctcctcttttggggcaagttcggaatcctcaccctcaagtacaagtccagccacaccaacaagagaattatcctgctcctggtggccggcctggtcctcaccgtgatcgtggtggtcggcgccattctgctcattcccggagagaagcctgtcaaaaacgcctccggcctcggactcattgtgatcagcaccggaatcctgatcctcctgcagtacaacgtgttcatgacagctttcggaatgacctccttcaccatcgctatcctgattacccaggtgctgggatatgtgctggccctcgtgggactgtgtctgtgcatcatggcttgcgaacccgtccatggccctctcctgatctccggcctgggcatcatcgccctcgctgagctgctcggactggtgtacatgaaattcgtggcctccaaccaaagaaccatccagcctcccaggaacagaggcggatccggccagtgcacaaactatgccctgctcaagctggccggcgatgtggaaagcaatcccggccctatgatcaggggaagggcccctaggacaaggcctagccctcctccccctctcctccctctcctgagcctgagcctgctgctgctgtcccctaccgtgagaggcgactgcggccccccccccgacattcctaacgccagacctattctgggcaggcatagcaaatttgccgaacaaagcaaggtcgcctacagctgcaacaacggcttcaagcaggtccccgacaaaagcaacattgtcgtgtgcctcgagaacggccaatggtccagccatgaaacattctgcgaaaaaagctgcgtggctcccgagaggctgtccttcgcctccctgaaaaaggaatatctgaatatgaatttcttccctgtgggcaccattgtggagtatgagtgtaggcccggattcagaaagcagccccctctgcccggaaaagctacatgtctggaagatctggtgtggagccccgtggctcaattctgcaaaaagaagagctgtcccaatcccaaggacctcgataatggccacatcaacattcctaccggaatcctgttcggcagcgaaattaactttagctgcaatcccggatataggctcgtcggcgtcagcagcaccttctgttccgtgaccggaaacaccgtggactgggacgacgaattccccgtctgtaccgagattcattgccctgagcctcctaagattaacaatggcatcatgagaggcgaaagcgacagctacacctattcccaggtcgtcacatatagctgcgacaagggattcatcctggtcggcaatgctagcatctattgcaccgtgagcaaatccgatgtgggacaatggagcagcccccctcccaggtgcatcgaaaagagcaaagtgcccaccaaaaaacccacaatcaacgtgcctagcacaggcacaccttccacccctcaaaaacctacaaccgagtccgtgcccaatcctggagatcagcccacccctcagaaaccttccacagtcaaggtgagcgccacccagcatgtccctgtgaccaagacaacagtcagacatcctatcagaacctccaccgacaaaggagaacctaacaccggcggagacaggtacatttacggacacacctgtctgatcacactgacagtgctccatgtcatgctgtccctgatcggatacctcacaggctccggcgtgaaacaaaccctgaacttcgatctgctgaaactcgccggagacgtcgagtccaaccccggccctatgacagagtacaagcccacagtgagactcgccaccagggacgacgtccccagagctgtcagaaccctggctgccgcttttgccgattatcccgctaccaggcataccgtcgatcccgacaggcatatcgagagggtcaccgagctccaggaactgtttctgaccagagtcggactcgacattggcaaggtgtgggtcgccgacgacggagccgccgtggctgtgtggaccacacccgaaagcgtcgaagccggagccgtgttcgctgagattggacccagaatggccgagctgagcggctccaggctcgccgctcaacagcagatggagggcctcctggcccctcacagacccaaagaacccgcctggttcctggccacagtgggagtgtcccccgaccatcagggaaagggcctcggcagcgctgtggtcctccctggcgtggaggccgccgaaagggctggagtgcctgccttcctggaaacaagcgctcccagaaacctgcccttctatgagaggctcggctttaccgtgacagccgacgtcgagtgtcccaaggacagggccacctggtgtatgacaaggaaacccggcgcttgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 5 - DNA, 4026 bp, Artificial Sequence
  synthetic construct
ggccagagagccactcccccagcgccagaccaccctcttcctcactcccccacctcaccccctcacaggtgcctcccaggccatcagggcccaaccacccctaaacaaatgggttctcggcccctcgtggctggaggtgggttctctcaccattcccagcctaaggctccatccccatgctggcagctgttcaaccatgtctagagagatccactgtcccagacagcacctcagggtcccccgtcctgcctggaaccctgtaggaaactccacaaaccgccgccattctgtccacacccctacaggagccccaaccctctccccacatccaggcttccctcccagacccctcatccctgcccgcacggtgcctgagggggccttcttgggcagcgcctaagcaagcccccagcacccttcggccccttcaaggcacacaggccccctttccacccagcctcaggaaaccacctgtgtcctccaacgacaggtcccagcctcccagcctttgccttgcctgttcctctccctggaactctgccccgacacagaccctccccagcaagcccgcaggggcacctcccctgcccccagacaccctgtgcccgtcagttcatccccagcagaggccctcaccaggcacacccccatgctcacacctggccgcaggcctcagcctccctgagggccccacccagcccgcgtctggccagtggtgcgtgcaaagcccctcacccagattaattaaataacttcgtatagcatacattatacgaagttatgcggccgccagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctcgagataacttcgtatagcatacattatacgaagttatccgcggttgattgaagtgctgagtaaatggttgcaaacataggtctacatttttcaaatcattcaccataaatttgaattatttattaattacactcgaataaagcaataaagaaactgatgagataatatttgactgaattgcagcaataaatagatcgatattaacacaaggaatataactgacttccaaaaacatacacatgaaccgtggttcactctgcgtatttaggtaaataacagaaagttgtcataacagatggggaatcctgcagacttcactaggcatgggccatgctgccctggagttgtctcaggggagctgcctcctccagaggttagagcacaggcccaggtaataggactaaatttttagatgtgttatcttagacacactgcacaactgctgtgttctctatgtaaattatctcctgtaaaatataacattgaagcctgcattaaatatattgtgtaaatatgtaagaataaaagaaagttatgagagctaagtgttaatcaaggcacaagcatataagatataactatattttcctgaatgatggaattactaccagtctcccccaggacacttcatctgccctgagcccagcctctcctcagatgtcccacccagagcttgctatatagtgggggacatgcaaatagggccctccctctactgatgaaaaccagcccagccctgaccctgcagctctgggagaggagcccagcactagaagtcggcggtgtttccattcggtgatcagcactgaacacagaggactcaccatggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtgcagtctgggggaggcgtggtccagcctgggaggtccctgagactctcctgtgtagcctctggattcacgttcagtacctatgccatgcactgggtccgtcaggctccaggcagggggctggagtgggtggcagttatctcatacgatggaaattataaatactatgcagactctgtgaagggccgattctccatctccagagacaattccaacaacacgctgcatctagaaatgaacaccctgagaactgaggacacggctttatattactgtgcgaaagactcccaactgcgatcactcctctattttgaatggttatcccagggatattttgacccctggggccagggaaccctggtcaccgtcacctcaggttaagaatggccactctagggcctttgttttctgctactgcctgtggggtttcctgagcattgcaggttggtcctcggcccatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagc

SEQ ID NO: 6 - DNA, 4125 bp, Artificial Sequence
  synthetic construct
tgttgaatagtttggtcatctcagaaaagagaggctgggaagaataaggatgaactatacatatcaacctttcagaatatatttcagtaaacttcctagagcccatgacagaagcattctcttctaccatattccctttccatccatgcatataaccatctgtacttctatccacccatctgttgatccttcctcaagtcataaggctcattgacagcccttctacaccaaacaacaccaactccagagaaaaacctgttcatatgcctggacgtatgaatgtccaccttcactaattccatagaaatgtattagcatttccatgtacctggctctctatgaagatttgcagataacatagtcaacaagacagaactctctacctcaaggagctcctagagtagtgtataaacaaataaccagaaattgcaacatggagaaatggagctgagaggagtgcctacagagctctacgtaagcacacagaaggggtgcctaacacatattggaggccagagaagttatacccctcctgcccaaacattaaccaacctgggaattacctaacatcaacagtgtcttcacacgtcattccaatgctctagccctctgattgggataccatgggttagtgagctctcacatgggaatctatcacttttttatcccctctttgtttggggttacttgtttataataaagcaataacaataaacctctatgtcaacatcactgtgaacagcaaatgaaatattgtgtataatccactttgtttaattaaataacttcgtatagcatacattatacgaagttatgcggccgcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgcctggggagcctggggactttccacacctcgagataacttcgtatagcatacattatacgaagttatccgcggttgtgtgtgtgtgaagctcctattataataatgagaatactttgattcactcagttaaagttttcccctgatttattgtgtacatacaatgaaggatcaagaaagagaaatttttaaatggaagcattagccagacaagtttgacctcacagttttactaggggatatatcacctagttttggatctatttctaacatcttaacattgtgaaaagagtcttgggaaactggttaaatcccaaagaatgctgcaataggaggttggcccttatgagttatttaatatcttgagctgccttcggaaaatgttgctgagcaggcattgaagagtatcgataaaatttattgagaatttgtttattatgattaacagaggtaaaagccagtatattactgattaatataggtaaaaggcagttaagaaattgggaatgctttctcttctgctttcttctacgatgcacaaggcgtttcacatttatgcccctatgaaaattactaggctgtcctagtcattagatctttcagcagtttgtagttttagagcttctaagttgacttctgtcttttctattcatacaattacacattctgtgatgatatttttggctcttgatttacattgggtactttcacaacccactgctcatgaaatttgcttttgtactcactggttgtttttgcataggcccctccaggccacgaccagctgtttggattttataaacgggccgtttgcattgtgaactgagctacaacaggcaggcaggggcagcaagatggtgttgcagacccaggtcttcatttctctgttgctctggatctctggtgaggaattaaaaagtgccacagtcttttcagagtaatatctgtgtagaaataaaaaaaattaagatatagttggaaataatgactatttccaatatggatccaattatctgctgacttataatactactagaaagcaaatttaaatgacatatttcaattatatctgagacagcgtgtataagtttatgtataatcattgtccattactgactacaggtgcctacggggacatccagatgacccagtctccagactccctggctgtatctctgggcgcgagggccaccatcaactgcaagtccagccagagtgtcaccttcaactataagaactacttagcttggtaccagcagaaaccaggacagcctcctaaagtgctcatttactgggcatctgcccgggaatcaggggtccctgaccgattcagtggcagcgggtctgggacagatttcactctcaccatcagcagcctgcaggctgaagatgtggctgtttattactgtcagcaacattataggactcctccgacgttcggccaagggaccaaggtggagatcaaacgtaagtacatctgtctcaattattcgtgagattttagtgccattgtatcatttgtgcaagttttgtgatattttggttgaataaacctggtgacccagaagtaaatagcaggacaccagaaaatgaacttaaaaagctgagcaaatagacgaatcattgggtttgagaggagaataggattcatgggggaaatggggaagaaatagctagatttttctctgaacaagcagcctatctcatatgattggcttcaagagaggtttttgttgaggggaaagggtgagatccctcactgtggctcactttcggcggagggaccaaggtggagatcaaacgtaagtgcactttcctaatgctttttcttataaggttttaaatttggagcgtttttgtgtttgagatattagctcaggtcaattccaaagagtaccagattctttcaaaaagtcagatgagtaagggatagaaaattagttcatcttaaggaacagccaagcgctagccagttaagtgaggcatctcaattgcaagattttctctgcatcggtcaggttagtgatattaacagcgaaaagagatttttgttaaggggaaagtaattaagttaacactgtggatcaccttcggccaagggacacgactggagattaaacgtaagtaatttttcactattgtcttctgaaatttgggtctgatggccagtattgacttttagaggcttaaataggagtttggtaaagattggtaaatgagggcatttaagatttgccatgggttgcaaaagttaaactcagcttcaa

(19) United States
(12) Patent Application Publication
(10) Pub. No.: US 2020/0095543 A1
(43) Pub. Date: Mar. 26, 2020

(54) Cells and Methods of Uses and Making the Same

(71) Applicant: Washington University, St. Louis, MO (US)

(72) Inventors: Deepta Bhattacharya, St. Louis, MO (US); Hannah Pizzato, St. Louis, MO (US); Yinan Wang, St. Louis, MO (US); Derrick Callahan, St. Louis, MO (US)

(21) Appl. No.: 16/495,318

(22) PCT Filed: Mar. 30, 2018
(86) PCT No.: PCT/US2018/023288

§ 371 Date: Sep. 18, 2019

Related U.S. Application Data
(60) Provisional application No. 62/473,564, filed on Mar. 20, 2017.

(51) Int. Cl. C12N 5/0735; C07K 16/08; A61K 35/545

(52) U.S. Cl. CPC C12N 5/0606; C07K 16/08; A61K 35/545; C07K 2317/21; C12N 2506/02; C12N 2510/00; C07K 2317/76

(57) ABSTRACT

Among the various aspects of the present disclosure is the provision of a genetically engineered stem cells, plasma cells, B cells to avoid immune rejection within a host, and methods of making the same and uses thereof.

### CROSS-REFERENCE TO RELATED APPLICATIONS

This application claims priority from U.S. Provisional Application Ser. No. 62/473,564 filed on 20 Mar. 2017, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to cellular therapies.

### BACKGROUND OF THE INVENTION

Vaccines against devastating infectious diseases represent some of mankind's greatest medical breakthroughs. The underlying principle of vaccines is the establishment of immunity using attenuated or inactivated vectors that mimic the natural pathogen without causing the systemic damage a live infection can trigger. Thus the prerequisite for all successful vaccines to date is that the human immune system must be inherently capable of eventually generating adaptive immunity when faced with the natural live infection. In cases like polio, the damage achieved before adaptive immunity can control the natural virus is unacceptable; nevertheless, lasting immunity is ultimately achieved and protects against subsequent infections.

Currently, however, many of the most problematic infectious diseases worldwide cannot be effectively controlled by the human immune system and do not elicit lasting immunity against re-infection. For example, by virtue of its mutability, HIV establishes chronic infections that cannot be cleared and are ineffectively controlled without the assistance of antiviral drugs. Dengue virus infection leads to lasting immunity against re-infection by the same serotype, but in fact causes far more severe disease if the individual is re-infected with a different Dengue serotype than if he/she were completely naïve. Similarly, influenza strains continuously alter themselves genetically; thus immunity to a given strain rarely affords complete protection against all subsequent influenza viruses that circulate in time and space. Natural infection with malaria does not necessarily lead to lasting immunity, as the same individual can be re-infected many times over the course of a lifetime. Pathogens like these can pose a conundrum: how can vaccines designed to mimic the natural pathogen elicit immunity when the immune system is intrinsically incapable of generating broad and effective immunity when faced with the actual infection? Innovative alternate strategies, such as structure-guided sequential immunizations, gene therapy, and cell-based therapy have all been proposed, though the latter approach is the least developed.

Additionally, the field of cell therapies requires advances in immune suppression technologies or cell technologies which avoid immune rejection. Advances in understanding immune surveillance and genetic manipulations are enabling the engineering of cellular modification to tailor cells to avoid immune rejection.

### SUMMARY OF THE INVENTION

Among the various aspects of the present disclosure is the provision of generating plasma cells, genetically engineered stem cells, methods of making plasma cells and genetically engineered stem cells to avoid immune rejection within a host, and uses thereof.

An aspect of the present disclosure includes a method of differentiating human embryonic stem cells (ES cells) into transplantable plasma cells that produce a therapeutic agent (such as broadly neutralizing antibodies, proteins, or enzymes). In some embodiments, the method comprises generating progenitor cells (e.g., hemogenic progenitor cells) and differentiating the progenitor cells into B cells.

In some embodiments, differentiating the progenitor into B cells comprises: expressing a B lineage-promoting genetic factor driven by an inducible promoter (optionally, PAX5, EBF1, FOXO1A, BCL11A, TCF3, IKZF1, IRF4, IRF8, or SPI1), co-culturing the progenitor with (i) a cytokine selected from one or more of the group consisting of: Flt3L, SCF, and IL-7, and (ii) MS5 stromal cells, for a period of time sufficient for promoting B lymphopoiesis (optionally, for about 20 days); or activating the progenitor with a stimulus, resulting in B cell differentiation.

In some embodiments, the genetic factor is optionally selected from one or more of the group consisting of PAX5, EBF1, FOXO1A, BCL11A, TCF3, IKZF1, IRF4, IRF8, or SPI1.

In some embodiments, the stimulus is selected from doxycycline or tetracycline.

In some embodiments, the hemogenic progenitor is a hemogenic endothelial cell; the hemogenic progenitor is transduced (optionally transduced with a lentiviral vector) encoding constitutively expressed rTTA-T2A-GFP; a stimulus-inducible transcriptional activator linked to a reporter by a ribosome skipping 2A sequence; or the genetic factors are selected from one or more of the group consisting of PAX5, EBF1, FOXO1A, BCL11A, TCF3, IKZF1, IRF4, IRF8, or SPI1 driven by an inducible promoter, resulting in a transduced cell, the transduced cell constitutively express rTTA and GFP, but express the genetic factors only upon stimulus (e.g., doxycycline) treatment.

In some embodiments, modified RNA is used to transiently introduce factors only when they are needed for differentiation and lineage commitment.

In some embodiments, human ES cells are nucleofected with a pathogen-specific, antibody gene-encoding cassette.

In some embodiments, the pathogen specific antibody cassette comprises VDJ and VJ sequences selected from one or more of the group consisting of flu antibody, HIV antibody, or flavivirus antibody.

In some embodiments, the specific antibody cassette comprising VDJ and VJ sequences are selected from one or more of the group consisting of F16, VRC07, 10E8, N6, 3BNC117, EDE1, and 010.

In some embodiments, Cas9, guide RNAs (gRNAs), ZFN, or TALENs are used to target the endogenous immunoglobulin heavy chain and kappa light chain loci.

Another aspect of the present disclosure provides for a method of generating a long-lived plasma cell with enhanced glucose uptake comprising administering IFNγ or IL-4 to B cells derived from human ES cells.

In some embodiments, the long-lived plasma cell has elevated antibody secretion and elevated mitochondrial pyruvate for respiration.

Yet another aspect of the present disclosure provides for a method of generating a long-lived plasma cell comprising: providing primary tonsillar naïve B cells; or introducing IL-4 or IFNγ to the primary tonsillar naïve B cells.

Yet another aspect of the present disclosure provides for a method of generating a long-lived plasma cell comprising: providing 3T3 fibroblast cells engineered to express CD40L and BAFF; or culturing the cells in the presence of IL-21.

Yet another aspect of the present disclosure provides for a method of treating a subject having a virus comprising administering a therapeutically effective amount of a plasma cell to a subject, wherein the plasma cell expresses sequences from an antibody broadly neutralizing the virus.

Yet another aspect of the present disclosure provides for a method of treating a subject in need of enzyme replacement therapy comprising administration of a plasma cell engineered to secrete an enzyme.

In some embodiments, the plasma cell is engineered to secrete enzymes through genetic replacement or IRES knockins downstream of Aβ genes.

Yet another aspect of the present disclosure provides for a B cell or plasma cell generated by the previously described methods.

Yet another aspect of the present disclosure provides for a method of treating an autoimmune disease or cancer comprising a plasma cell expressing an immunotherapeutic agent, such as rituxan or eculizimab.

Yet another aspect of the present disclosure provides for a method of treating a neurodegenerative disorder comprising administering a therapeutically effective amount of a plasma cell expressing an immunotherapeutic agent, to a subject.

In some embodiments, the neurodegenerative disorder is Alzheimer's disease or the immunotherapeutic agent is aducanumab.

Yet another aspect of the present disclosure provides for a genetically engineered stem cell comprising: (i) modulated expression of one or more HLA-I and HLA-II relative to a wild-type stem cell; or (ii) constructs encoding genes leading to the evasion of complement fixation, the evasion of NK cell recognition, or the evasion of phagocytosis.

In some embodiments, the genetically engineered stem cell comprises modulated expression of one or more immune evasion factors relative to a wild-type stem cell. In some embodiments, the modulated expression of the immune evasion factors comprises increased expression of the immune evasion factors.

In some embodiments, the immune evasion factors are inserted into a safe harbor locus of at least one allele of the cell. In some embodiments, the safe harbor locus comprises an AAVS locus.

In some embodiments, the immune evasion factors inhibit immune rejection or promote immune evasion.

In some embodiments, the immune evasion factors are selected from the group consisting of HLA-E, HLA-G, CD46/Crry, CD47, or CD55.

In some embodiments, the stem cell is an embryonic stem cell. In some embodiments, the stem cell is a pluripotent stem cell. In some embodiments, the stem cell is hypoimmunogenic. In some embodiments, the stem cell is a human stem cell.

In some embodiments, the genetically engineered stem cell comprises genetic modifications in encoding genes selected from one or more of the group consisting of: β2 microglobulin, TAP1, CD74, CIITA, and ligands of NKG2D.

In some embodiments, ligands of NKG2D are optionally selected from one or more of the group consisting of MICA, MICB, Raet1e, Raet1g, Raet11, Ulbp1, Ulbp2, and Ulbp3.

In some embodiments, the genetic modification comprises an inactivating mutation.

In some embodiments, the genetically engineered stem cell comprises enhanced expression of one or more of HLA-E and HLA-G.

In some embodiments, the genetically engineered stem cell comprises enhanced expression of one or more of HLA-E and HLA-G relative to a wild-type stem cell.

In some embodiments, (i) β2 microglobulin and TAP1-encoding genes are genetically modified to eliminate HLA-I expression and prevent direct recognition by allogeneic CD8+ T cells; (ii) CD74-encoding genes are genetically modified to eliminate HLA-II expression; (iii) NKG2D ligand encoding genes are genetically modified to evade natural killer cell recognition; or (iv) the stem cell exhibits reduced immunogenicity.

In some embodiments, (i) at least one immune evasion gene selected from the group consisting of: HLA-E single chain trimer, HLA-G single chain trimer, K-b single chain trimer, CD46/Crry, CD55, and CD47; (ii) at least one immune evasion gene inhibiting the pathway or cell type selected from the group consisting of NKG2A+NK cells, ILT2/KIR2DL4+NK cells, Ly49C+NK cells, complement/C3b and C4b, complement/C3 convertase, complement/C9, and phagocytosis; or (iii) optionally, at least one suicide gene.

In some embodiments, (i) an immune evasion gene or immune evasion factor is delivered to a safe harbor locus protected from silencing, the immune evasion factor is selected from one or more of the group consisting of: HLA-E single chain trimer, HLA-G single chain trimer, K-b single chain trimer, CD46/Crry, CD55, and CD47; (ii) the suicide gene is selected from one or more of the group consisting of: iCasp9, HSV thymidine kinase, cytosine deaminase, and E. coli nitroreductase; or (iii) the suicide gene is selected from a suicide gene with an inducing drug selected from the group consisting of AP1903, ganciclovir, 5-fluorocytosine, and CB1954.

Yet another aspect of the present disclosure provides for a genetically engineered stem cell comprising genetic modifications in encoding genes that evade recognition by one or more immune cells selected from the group consisting of: CD8⁺ T cells, CD4⁺ T cells, and NK cells.

In some embodiments, the genetic modification is in an encoding gene selected from one or more of the group consisting of: β2 microglobulin, TAP1, CD74, CIITA, and ligands of NKG2D, wherein the ligands of NKG2D are optionally selected from one or more of the group consisting of: MICA, MICB, Raet1e, Raet1g, Raet1I Ulbp1, Ulbp2, and Ulbp3.

In some embodiments, the genetic modification comprises an inactivating mutation.

Yet another aspect of the present disclosure provides for a genetically engineered stem cell comprising modulated expression of one or more immune evasion factors relative to a wild-type human stem cell.

In some embodiments, the modulated expression of the immune evasion factors comprises increased expression of the immune evasion factors.

In some embodiments, the immune evasion factors are inserted into a safe harbor locus of at least one allele of the cell.

In some embodiments, the safe harbor locus comprises an AAVS locus.

In some embodiments, the immune evasion factors inhibit immune rejection or promote immune evasion.

In some embodiments, the stem cell is an embryonic stem cell. In some embodiments, the stem cell is a pluripotent stem cell. In some embodiments, the stem cell is hypoimmunogenic. In some embodiments, the stem cell is a human stem cell.

In some embodiments, the immune evasion factors are selected from one or more of the group consisting of HLA-E, HLA-G, CD46/Crry, CD47, and CD55.

In some embodiments, the genetically engineered stem cell comprises modulated expression of one or more HLA-I and HLA-II relative to a wild-type stem cell.

Yet another aspect of the present disclosure provides for a method for making a genetically engineered stem cell comprising delivering a construct to a safe harbor locus, and encoding genes that lead to evasion of complement fixation, NK cell recognition, or phagocytosis. In some embodiments, the gene leading to evasion of complement fixation is selected from one or more of the group consisting of CD46/Crry, CD55, and CD59. In some embodiments, the gene leading to evasion of NK cell recognition is selected from one or more of the group consisting of HLA-E and HLA-G. In some embodiments, the gene leading to evasion of phagocytosis is CD47.

Yet another aspect of the present disclosure provides for a method of treating a subject in need thereof with a genetically engineered stem cell or a genetically engineered stem cell.

In some embodiments, the stem cell is transplanted into a subject; a tissue of a subject is destroyed by an autoimmune disease; a tissue of a subject destroyed by an autoimmune disease is regenerated; pancreatic cells or oligodendrocytes are regenerated; the subject has type I diabetes, multiple sclerosis, a pathogen or an infectious disease; humoral immunity is generated; or antibody-mediated immunity is generated.

In some embodiments, gene editing is used to ablate the CD74 or CIITA, a transcription factor required for expression of HLA-II, wherein the genetically engineered stem cells retained hematopoietic potential, and derivative monocytes lacked detectable expression of HLA-II.

In some embodiments, host immunosuppression is absent.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1B shows the generation of definitive hematopoietic precursors from hES cells. (A) Schematic of hES cell differentiation protocol. (B) Example of differentiation. H1 hES cells were differentiated into mesoderm and then into definitive hemogenic endothelium for 8 days. These cells were co-cultured with OP9-DLL4 cells for an additional 3 weeks to generate committed CD7+ CD5+ CD4− CD8− ‘DN’ T cells.

FIG. 2A-FIG. 2B shows doxycycline-induced Pax5 expression promotes B cell differentiation from hES-derived progenitors. (A) Schematic of lentiviral vector used to transduce hemogenic endothelium. (B) Representative data demonstrating B cell differentiation of doxycycline-treated lentivirally transduced cells after 25 days of co-culture with MS5 stromal cells.

FIG. 3A-FIG. 3C shows the targeting of influenza-specific antibody genes into endogenous immunoglobulin loci. (A) Schematic of IgH targeting strategy. (B) PCR screening of genomic DNA from zeocin-resistant clones or K562 cell line positive control transfectants. Similar strategies were undertaken for Igκ targeting. (C) PCR screening of genomic DNA hES clones carrying influenza antibody genes for drug resistance cassettes after transfection of Cre recombinase.

FIG. 4 shows IFNγ promotes glucose uptake during in vitro plasma cell differentiation. Naïve B cells were cultured on NIH 3T3 cells expressing CD40L and BAFF for 3 days in the presence or absence of the indicated cytokines. Cells were then incubated for 30 minutes with 50 μM 2NBDG, a fluorescent glucose analog, and analyzed by flow cytometry.

FIG. 5A-FIG. 5B shows engraftment and persistence of primary human bone marrow plasma cells in ossicles. 1-5×10⁶ CD138-enriched human bone marrow plasma cells were injected into pre-formed human ossicles in NSG mice. Serum ELISAs at days 7 and 28 (A) and flow cytometric analysis (B) at Day 28 was performed. Lower limit of detection for (A) was 3 pg/ml.

FIG. 6A-FIG. 6E shows the generation of an HLA-deficient hES cell line. (A) Schematic of genome editing workflow. Cas9 and three gRNAs targeted to genes essential for HLA expression were nucleofected into H1 hES cells. Two rounds of subcloning and MiSeq analysis yield clonal mutant cell lines. (B) Example of Miseq analysis of targeted genes. Frameshift mutations were introduced in 5 of 6 alleles. (C) Wild-type or HLA-KO hES cells were stained for HLA-I expression with or without IFNγ-treatment. HLA-I expression was absent in β2m- and TAP1-deficient cells. (D) Monocytes and dendritic cells derived from HLA-KO hES cells or controls were mixed with allogeneic T cells labeled with CFSE. CFSE dilution was quantified 4 days later. (E) Monocytes and dendritic cells were derived from HLA-KO hES cells or from control hES cells, and HLA-II expression was measured by flow cytometry.

FIG. 7A-FIG. 7B shows immune evasion genes to be delivered to AAVS locus. (A) Schematic of AAVS targeting constructs to evade human (top) or mouse (bottom) immune recognition. (B) AAVS constructs were transfected into HLA-KO hES cells and selected for puromycin or neomycin resistance. Expression of immune evasion genes was quantified by flow cytometry.

FIG. 8A-FIG. 8C shows AAVS constructs mediate immune evasion. (A) CHO cells were transfected with construct 1 (SEQ ID NO: 1) or 2 (SEQ ID NO: 2) in FIG. 7 and analyzed by flow cytometry. (B) Stable transfected CHO cells with construct 1 (top row) or construct 2 (bottom row) from FIG. 7 were tested for complement deposition. (C) 721.221 cells transfected with construct 1 in FIG. 7 were cultured with primary human NK cells. NK cell degranulation was measured as a function of CD107a expression.

FIG. 9A-FIG. 9B shows survival of HLA-KO grafts in humanized mice. (A) Robust engraftment of unconditioned NSG-W41 mice with cord blood CD34+ cells. Example of splenic chimerism 20 weeks post-transplant of 10⁵ CD34+ cells from cord blood. (B) HLA-KO, but not wild-type hES cells, form teratomas in humanized NSG-W41 mice.

FIG. 10 illustrates the development of cell-based therapies and strategies to engineer universally transplantable cells that provide durable antibody-mediated immunity showing that a stem cell-based therapy can generate immunity to mutable viruses.

FIG. 11A-FIG. 11B demonstrates successful deletion of MICA and MICB in HLA-KO cells. (A) Two separate gRNAs targeting MICA and MICB were transfected, and clones were sequenced for frameshift mutations. Clones B05, H10, and F01 carry frameshift mutations in MICA and MICB in one allele. (B) Targeting of a MICA-MICB fusion allele with gRNAs. On the other chromosome, an in-frame fusion was observed between MICA and MICB in clones B05, H10, and F01, caused by deletion of intervening sequences. This fusion allele was retargeted with a gRNA, and 5 clones carrying frameshift mutations were isolated.

### DETAILED DESCRIPTION OF THE INVENTION

The present disclosure is based, at least in part, on the discovery that (1) differentiating human pluripotent stem cells into transplantable antibody-or enzyme-secreting plasma cells and (2) mutations in genes (e.g., using CRISPR/Cas9) that encode ligands of NKG2D (evading natural killer cell recognition and resulting in a substantially non-immunogenic or minimally immunogenic human pluripotent stem cell for transplantation) and expression of genes that prevent complement deposition can eliminate major determinants of immunogenicity from human pluripotent stem cells. Together, these allow for scalable off-the-shelf therapies for autoimmune disorders, neurodegenerative diseases, cancer, and infectious disease as well as the general application of ES cell based therapeutics using cells altered to avoid immune rejection

As described herein, methods and targets used to modify human pluripotent stem cells so that they evade recognition of several arms of the immune system have been developed. The present disclosure provides for methods to generate a minimally immunogenic donor pluripotent stem cell line that can be used without host immunosuppression and as a source of ‘off-the-shelf’ therapies for regenerative medicine as well as cells generated by such methods.

One aspect of the present disclosure provides for a scalable input of cells altered to avoid immune rejection. Commercially viable cell-based therapies can require a scalable input of cells to reduce costs of treatment and to standardize manufacturing and which can serve as an allogenic source of cells for used to create cellular therapies.

Described herein is the development of the individual steps for scalable cellular therapies for infectious disease. These steps can be assembled into an integrated approach to provide protection against infectious pathogens as well as used in other cellular therapy applications.

Minimally Immunogenic or Substantially Non-Immunogenic Human Embryonic Stem Cells

The generation of minimally immunogenic ‘universal’ donor hES cell lines has been a goal of many fields in regenerative medicine. Decades of research on transplantation have made it clear that the immunological barriers to engraftment are substantial, and it was not entirely clear how best to achieve a truly ‘universal’ line.

Described herein are substantially or minimally immunogenic hES cells for transplantation, in particular, stem cell-based immunotherapies for infectious disease. The creation of such a line for transplantation allows for scalable off-the-shelf cellular therapies. This is desired for most stem cell-based therapies being developed by private industry. Such a line can also facilitate regenerative medicine for tissues destroyed by autoimmunity, such as pancreatic β cells in type I diabetes and oligodendrocytes in multiple sclerosis.

As described herein is the ablation of several major determinants of immunogenicity.

Applications for Disease Treatment

The present disclosure provides for the production of a scalable source of off-the-shelf therapies, with applications ranging from immunotherapy, pancreatic beta cell replacement, or restoration of oligodendrocytes for multiple sclerosis.

Different types of antibodies mediate long-term immunity against viruses that have been encountered previously, versus those that have mutated since the first exposure. Described herein is the identification of signals that lead to these different antibodies and define how they protect against pathogens.

Described herein are compositions and methods for providing durable immunity against globally relevant pathogens. Infectious disease contributes to nearly ⅓ of all deaths, and as such is the leading cause of death worldwide. Many of the most problematic infectious diseases have proven recalcitrant to vaccination despite decades of research on the microorganisms that cause these illnesses. Thus, new approaches are needed if successful vaccines are to be developed. Efforts to develop vaccines against HIV serve as useful examples. A small proportion of the HIV-seropositive population develops exceptionally potent antibodies capable of neutralizing >90% of clinical HIV isolates. Similar rare but broadly neutralizing antibodies have been discovered for influenza and Dengue viruses. Yet many of these antibodies are structurally unusual with an exceptionally high number of somatic mutations. Thus, it is not clear how to generate these types of responses in the general population.

Several creative possibilities have been proposed and explored experimentally. First, it has been proposed that through structure-based design of HIV glycoprotein immunogens and sequential vaccinations, the antibody response can be guided toward broadly neutralizing characteristics. Several studies have shown the feasibility of this approach using either immunoglobulin knockin mice or animals that carry more diverse repertoires. Yet these studies are still in the early stages of mouse models, and it remains to be seen whether this strategy will translate to human vaccines. Practically, it is unclear if similar resources devoted to the structural design of HIV epitopes would be available for other problematic pathogens. In the second approach, adeno-associated virus (AAV) vectors are used to deliver broadly neutralizing antibody genes intramuscularly for heterologous expression. Both mouse and primate models have shown promising results, leading to clinical trials. Although largely safe, the major problem revealed by previous studies is that the duration of heterologous gene expression can be quite transient unless the vector is delivered to immune-privileged sites. This is likely due to widespread pre-existing immunity and CD8⁺ T cell responses against AAV in humans. In a third approach, lentiviral-based gene therapy is used to modify hematopoietic stem cells (HSCs) such that they express broadly neutralizing antibodies. Upon autologous transplantation, HSC-derived genetically modified B cells and plasma cells can be formed to secrete these neutralizing antibodies. This last approach has received much less attention due to concerns about practical feasibility. Hematopoietic stem cell transplants, even if autologous, have required cytoreductive conditioning to achieve efficient engraftment. Moreover, the random integration of lentiviral vectors in the host genome has been shown to trigger leukemia. Finally, the approach is not scalable, making the cost prohibitive. As described here, these problems are addressed in the latter approach by generating pathogen-specific B cells using scalable, minimally immunogenic inputs.

Tissue Regeneration.

There is a need for inexpensive and easy-to-manipulate stem cells that can be used to regenerate human tissues. Here is described a stem cell line (e.g., genetically engineered) that is minimally immunogenic and considered a ‘universal’ donor. These cells can be used without host immunosuppression, as a source of ‘off-the-shelf’ therapies for regenerative medicine.

Plasma Cells and Serum Antibodies

The generation of antibody-mediated immunity using pluripotent stem cells is described herein. It is believed that there are no antecedent papers on this topic or no other ongoing research projects elsewhere that are pursuing antibody-mediated immunity using pluripotent stem cells.

Described herein is the novel method of using human pluripotent stem cells to generate humoral immunity (see e.g., Example 1).

The component steps to achieve humoral immunity are well-accepted. The potency of serum antibodies in protection against pathogens has been known for over a century (see e.g., Behring, 1965). The existence of long-lived plasma cells and their responsibility for maintaining serum antibodies is widely accepted in the field (see e.g., Slifka, 1998 et al).

The generation of definitive hematopoietic precursors (DHP) from hES cells are well known; see e.g. Kennedy et al., 2012. Except as otherwise noted herein, therefore, the process of the present disclosure can be carried out in accordance with such processes. As described herein, definitive hematopoietic precursors (DHP) are first differentiated from ES using methods as described by Kennedy et al., Cell Reports, 2012; and Sturgeon et al., Nat Biotech, 2014.

Differentiation from DHP into B cells can be accomplished by activating PAX5 by any method known in the art. For example, PAX5 can be activated by gene transduction or activation by small molecule. As shown in Example 1, PAX5 was activated by transducing PAX5 into a target cells using a lentiviral vector. Additionally, accessory stromal cells (e.g., 3T3 fibroblasts) can be engineered to express a subset of these factors by retroviral transduction (e.g., BAFF and CD40L). Human embryonic stem cells (ES cells), as described herein, can be differentiated into plasma cells. As an example, the ES cells can be H1 human embryonic stem cells.

A progenitor cell is a biological cell that, like a stem cell, has a tendency to differentiate into a specific type of cell, but is already more specific than a stem cell and is pushed to differentiate into its “target” cell. For example, a progenitor cell can be a hemogenic progenitor cell (e.g., hemogenic endothelial cell) or hematopoietic progenitor cell.

As described herein, a definitive hematopoietic progenitor (DHP) cell can be differentiated into B cells using transcription factors or cytokines and ectopic expression of B lineage-promoting activator protein or genetic factors. These cytokines include IL-7, Flt3L, and SCF, ranging from 1-100 ng/μl. Other cytokines described herein are IFNγ or IL-4. A B lineage-promoting activator protein (also referred to as a B lineage-promoting transcription factor or a genetic factor) include PAX5, EBF1, FOXO1A, BCL11A, TCF3, IKZF1, IRF4, IRF8, and SPI1. Genetic factors can be expressed inducibly by lentiviral vectors, modified RNAs, or plasmid transfection.

Applications of Plasma Cells.

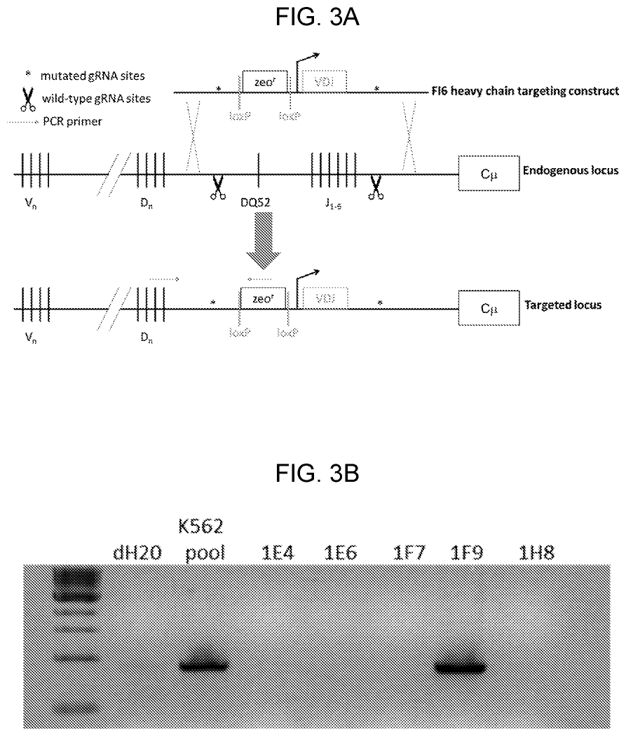

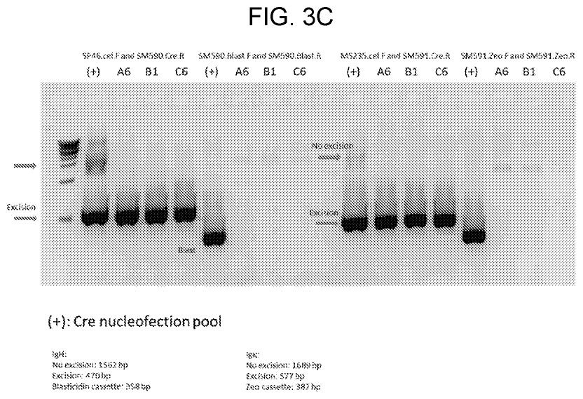

As described herein, plasma cells can be used as prophylaxis for infectious disease. For example, knocked in influenza antibody genes into the endogenous loci of hES cells were described (see e.g., Example 1, FIG. 3). These antibodies bind to a conserved part of the influenza virus and neutralize nearly all flu strains (see e.g., Example 1). As another example, plasma cells can be used for treatment of ongoing chronic infection such as HIV (see e.g., Example 1). As another example, plasma cells can be used for enzyme replacement therapy, for diseases associated with enzyme deficiency such as Hurler's syndrome (see e.g., Example 1) or Factor IX deficiency. As another example, plasma cells can be used for treatment of cancer (see e.g. Example 1). As another example, plasma cells can be used for treatment of autoimmune disease (see e.g., Example 1). As another example, plasma cells can be used for treatment of Alzheimer's disease (see e.g., Example 1). As another example, plasma cells expressing antibodies can be used for the treatment of a subject in need of antibody therapy (e.g., with an immunotherapeutic agent). For example, an immunotherapeutic agent can be an antibody. As an example, the immunotherapeutic agent can be rituxan, eculizimab, or aducanumab.

As described herein, the compositions and methods can be used to treat a neurodegenerative disease or disorder. For example, the neurodegenerative disease or disorder can be Alzheimer's disease, amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjögren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

As described herein, the compositions and methods can be used to treat cancer. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer (Head and Neck Cancer); Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer (Head and Neck Cancer); Leukemia; Lip and Oral Cavity Cancer (Head and Neck Cancer); Liver Cancer; Lung Cancer (Non-Small Cell and Small Cell); Lymphoma; Male Breast Cancer; Malignant Fibrous Histiocytoma of Bone or Osteosarcoma; Melanoma; Intraocular (Eye); Merkel Cell Carcinoma (Skin Cancer); Mesothelioma, Malignant; Metastatic Cancer; Metastatic Squamous Neck Cancer with Occult Primary (Head and Neck Cancer); Midline Tract Carcinoma Involving NUT Gene; Mouth Cancer (Head and Neck Cancer); Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer (Head and Neck Cancer); Nasopharyngeal Cancer (Head and Neck Cancer); Neuroblastoma; Non-Hodgkin Lymphoma; Non-Small Cell Lung Cancer; Oral Cancer, Lip or Oral Cavity Cancer; Oropharyngeal Cancer (Head and Neck Cancer); Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer (Head and Neck Cancer); Parathyroid Cancer; Penile Cancer; Pharyngeal Cancer (Head and Neck Cancer); Pheochromocytoma; Pituitary Tumor; Plasma Cell Neoplasm/Multiple Myeloma; Pleuropulmonary Blastoma; Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer (Head and Neck Cancer); Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic (Head and Neck Cancer); Stomach (Gastric) Cancer; T-Cell Lymphoma, Cutaneous; Lymphoma; Mycosis Fungoides and Sezary Syndrome; Testicular Cancer; Throat Cancer (Head and Neck Cancer); Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer); Urethral Cancer; Uterine Cancer, Endometrial; Uterine Sarcoma; Vaginal Cancer; Vascular Tumors (Soft Tissue Sarcoma); Vulvar Cancer; or Wilms Tumor.

As described herein, the compositions and methods can be used to treat an autoimmune disease or disorder. For example, the autoimmune disease or disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Bab disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum, Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse); Hypogammalglobulinemia, IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus, Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB, Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (POD); Paroxysmal nocturnal hemoglobinuria (PNH); Parry Romberg syndrome; Pars planitis (peripheral uveitis); Parsonnage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum, Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; Vogt-Koyanagi-Harada Disease; or Wegener's granulomatosis (or Granulomatosis with Polyangiitis (GPA)).

As described herein, plasma cells can be generated that express antibodies. For example, plasma cells can be generated from human ES cells nucleofected with a pathogen-specific, antibody gene-encoding cassette.

As described herein, the plasma cells can express antibodies specific for a virus. For example, the virus can be influenza, HIV, malaria, or a flavivirus (e.g., Dengue, Zika, West Nile virus, tick-borne encephalitis virus, yellow fever virus, cell fusing agent virus (CFAV), Palm Creek virus (PCV), Parramatta River virus (PaRV)). For example, the plasma cells can express antibodies specific for F16, VRC07, 10E8, N6, 3BNC117, EDE1, or C10.

Genetically Engineered Stem Cells by Genome Editing, an Immune Engineering Approach

As described herein, a genetically engineered pluripotent stem cell line was modified such that the cells evade recognition by several arms of the immune system. Alternation in such genes are typically preformed on a group of genes and targets, several of which have been described in the prior art. Key novel features of the present invention as added to prior art modification will be readily apparent to one skilled in the art.

Cells containing the novel modifications of the present invention, alone or in combination with those previously described, can evade recognition by CD8⁺ T cells, CD4⁺ T cells, NK cells, complement, or phagocytotic cells. Furthermore, the cells can contain inducible suicide genes and drug resistance cassettes. This allows for selective elimination of grafts in case of adverse effects, and facile drug selection in culture to identify clonal cell lines. Together the process allows for the generation of human pluripotent stem cells with significantly reduced immunogenicity for transplantation.

As described herein, it was discovered that disrupting specific immune receptors and introducing specific transgenes into human stem cells (i.e., modified by gene deletions and/or transgene (cDNA) insertions) can result in a universal donor pluripotent stem cell.

Provided herein is a genetically engineered stem cell wherein (i) β2 microglobulin and TAP1-encoding genes are genetically modified to eliminate HLA-I expression and prevent direct recognition by allogeneic CD8+ T cells; (ii) HLA-II expression is eliminated thus evading direct recognition by CD4+ T cells; or (iii) NKG2D ligand encoding genes are genetically modified to evade natural killer (NK) cell recognition.

Also provided herein is a stem cell line and a method of creating a stem cell line that evades recognition by the following immune cells: (i) CD8⁺ T cells (i.e., due to lack of MHC Class I expression with genetic modifications in the β2 microglobulin and TAP1-encoding genes); (ii) CD4⁺ T cells (i.e., due to lack of MHC Class II expression with genetic modifications in genes encoding CD74 and CITTA); and/or (iii) NK cells (i.e., due to genetic modifications in genes that encode ligands of NKG2D (e.g., MICA, MICB, Raet1e, Raet1g, Raet11, Ulbp1, Ulbp2, and Ulbp3)).

Also provided herein is a method for making a genetically engineered stem cell comprising delivering a construct to an AAVS locus in ES cells to express the following genes (or immune evasion factor) for the indicated purposes: (i) CD46/Crry, CD55, and CD59 resulting in evasion of complement fixation; (ii) HLA-E and HLA-G single chain trimers; K-b single chain trimer (shown in mouse) resulting in the evasion of NK cell recognition; (iii) CD47 resulting in evasion of phagocytotic macrophages; (iv) icasp9 and HSV thymidine kinase (death by AP1903 and ganciclovir, respectively) resulting in inducible suicide genes; and/or (v) puromycin and neomycin resistance cassettes resulting in drug resistance.

Also provided are methods of treating a subject with the genetically engineered pluripotent stem cell, wherein: the subject has an autoimmune disease, such as type I diabetes or multiple sclerosis, or the patient has an infectious disease (e.g., an infectious disease that destroys tissues); the subject has damaged tissue and the damaged tissue is regenerated by the genetically engineered stem cells (e.g., pancreatic cells, oligodendrocytes).

Genetic Modification to β2 Microglobulin and TAP1-Encoding Genes.

The present disclosure provides for genetic modifications in the β2 microglobulin and TAP1-encoding genes. This eliminates HLA-I expression and prevents direct recognition by allogeneic CD8+ T cells. As described herein the genetic modification can be an inactivating mutation.

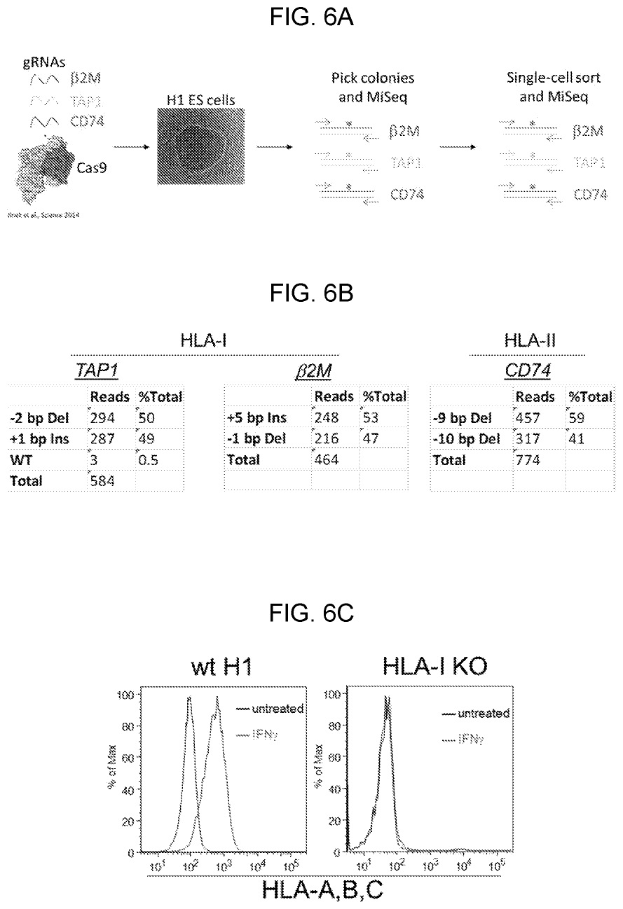

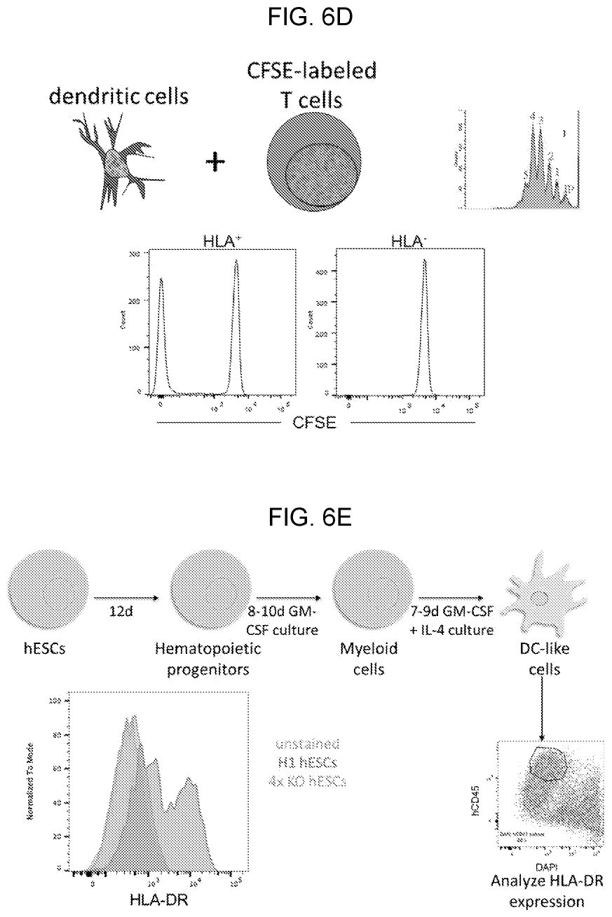

The present disclosure further provides for mutations in genes encoding CD74 and CIITA. This eliminates HLA-II expression and evades direct recognition by CD4+ T cells. Previous studies have described mutation of genes to separately prevent HLA-I and HLA-II expression. But, as described herein (see e.g., Example 2, FIG. 6), two additional genes have been identified to be targeted. For example, for preventing of HLA-I expression, the use of TAP1 mutations are described. The deletion of TAP1 in addition to β2M was shown to be important, as neither gene deletion by itself is sufficient to completely ablate HLA-I expression. Prior studies have demonstrated residual CD8+ T cell reactivity to TAP1-deficient cells, and β2M-deficient grafts can re-express HLA-I expression in β2M-sufficient hosts through the acquisition of serum β2M. As another example, for preventing HLA-II expression, mutating CD74 is described. Deleting CD74 in addition to CIITA was shown to be important, as some cell types can express HLA-II independently of CIITA. Depending on the primary mutations to ablate HLA-I and II, these additional genes can be ablated to prevent leaky HLA expression.

As described herein, an inactivating mutation can be any mutation in a gene resulting in reduction or elimination of expression of HLA-I or HLA-II (see e.g., FIG. 6). Inactivating mutations can include nucleotide insertions or deletions that change the reading frame and prevent translation of a functional protein. For example, a 2 base pair deletion in one allele of the TAP1 gene and a 1 base pair insertion in the other allele of the TAP1 gene led to a premature termination of translation and the absence of HLA-I expression (see e.g., FIG. 6).

The present disclosure further shows that these HLA-deficient cells generate teratomas in xenochimeric mice reconstituted with an allogeneic human immune system. As shown herein, it has been demonstrated that the cells lack expression of HLA-I and HLA-II. It was further shown that monocytes derived from these cells fail to stimulate allogeneic T cell proliferation. As further demonstrated herein, the modified cells were shown to evade rejection by mice reconstituted with a human immune system. It was also demonstrated herein that the AAVS targeting constructs properly express all intended genes and confer resistance to natural killer cell recognition and complement deposition.

It is presently believed that none of the previous approaches combined disruption of HLA-I and HLA-II. Moreover, the genes that were targeted in previous approaches are believed to be insufficient to mediate complete loss of HLA-I and HLA-II expression.

Prevention of Phagocytosis and NK Cell Activation.

The present disclosure further provides for mutations generated in genes that encode ligands of NKG2D to evade natural killer (NK) cell recognition. NK cells have many different modes of recognition, and NKG2D is the only activating receptor known to be expressed on all NK cells. Ablation of NKG2D ligands thus ablates reactivity by all NK cells, in contrast to other alternative strategies described in the prior art.

The present disclosure further provides for the design of and validation of constructs to be delivered to the AAVS locus in human ES cells. These constructs encode genes that lead to evasion of NK cell recognition (HLA-E and HLA-G single chain trimers), and phagocytosis (CD47 and HLA-G single chain trimers). Expression of these genes substantially reduces NK cell activation (see e.g., Example 2). These constructs simultaneously encode inducible suicide genes (e.g., icasp9 and HSV thymidine kinase) and drug resistance cassettes (e.g., puromycin and neomycin resistance). This allows for selective elimination of grafts in case of adverse effects, and facile drug selection in culture to identify clonal cell lines. Together the process allows for the generation of human pluripotent stem cells with significantly reduced immunogenicity for transplantation.

Prevention of Complement Deposition.

The present disclosure further provides for the design of and validation of constructs to be delivered to the AAVS locus in human ES cells. These constructs encode genes that lead to evasion of complement fixation (e.g., CD46/Crry, CD55, and CD59). The use of CD46/Crry, CD55, and CD59 expression to prevent classical and alternative complement deposition on cells is described herein (see e.g., Example 2). Prevention of classical and alternative complement deposition is believed to be critical for preventing antibody-dependent immune rejection.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus or activating agent (e.g., a doxycycline- or tetracycline-inducible promoter). A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a disease (e.g., an autoimmune disease, a tissue destroyed by an autoimmune disease, a pathogen, cancer, enzyme deficiency, or a neurodegenerative disease) with a cell-based therapy (e.g., differentiated progeny of a genetically engineered stem cell) in a subject in need thereof and administration of a therapeutically effective amount of a cell-based therapy, so as to treat the disease (e.g., pathogen, infectious disease) with a pluripotent stem cell while evading natural killer cell recognition.

Further, ES cells modified to avoid immune rejection using the methods of the present invention can be used to generate any other cell type currently being developed for use in patient therapy. Such differentiated cells are administered to a patient in need of such cells with reduced or without the need for immune suppressive agents.

Methods described herein are generally performed on a subject in need thereof. A subject in need of the therapeutic methods described herein can be a subject having, diagnosed with, suspected of having, or at risk for developing tissue damage, a pathogen, or an infectious disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a genetically engineered stem cell (e.g., a genetically engineered pluripotent stem cell) is, for example, that amount that would cause the desired therapeutic effect in a subject while minimizing undesired side effects. In various embodiments, an effective amount of a plasma cell derived from a genetically engineered stem cell described herein can substantially inhibit tissue damage, a pathogen, or an infectious disease, slow the progress of tissue damage, a pathogen, or an infectious disease, or limit the development of tissue damage, a pathogen, or an infectious disease.

According to the methods described herein, administration can be parenteral, pulmonary, oral, topical, intradermal, ossicle, intramuscular, intraperitoneal, intravenous, subcutaneous, intranasal, epidural, ophthalmic, buccal, or rectal administration.

When used in the treatments described herein, a therapeutically effective amount of a plasma cell derived from a genetically engineered stem cell (e.g., a genetically engineered pluripotent stem cell) can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit tissue damage, a pathogen, or an infectious disease, slow the progress of tissue damage, a pathogen, or an infectious disease, or limit the development of tissue damage, a pathogen, or an infectious disease. The genetically engineered stem cell can be minimally immunogenic, evading recognition of several arms of the immune system.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of progeny derived from genetically engineered stem cells can occur as a single event or over a time course of treatment. For example, progeny of genetically engineered stem cells can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for tissue damage, a pathogen, or an infectious disease.

The progeny of a genetically engineered stem cell can be administered simultaneously or sequentially with another agent, such as an antibiotic, an anti-inflammatory, or another agent. For example, the progeny of the genetically engineered stem cell can be administered simultaneously with another agent, such as an antibiotic or an anti-inflammatory. Simultaneous administration can occur through administration of separate compositions, each containing one or more of a progeny of a genetically engineered stem cell, an antibiotic, an anti-inflammatory, or another agent. Simultaneous administration can occur through administration of one composition containing two or more of progeny from genetically engineered stem cells, an antibiotic, an anti-inflammatory, or another agent. Differentiated progeny of genetically engineered stem cells can be administered sequentially with an antibiotic, an anti-inflammatory, or another agent. For example, cells derived from genetically engineered stem cells can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, intra-ossicle, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration.

Such kits can facilitate performance of the methods described herein. When supplied as a kit, the different components of the composition or for making the composition can be packaged in separate containers and admixed immediately before use. Components include, but are not limited to progenitor cells, progeny of universal pluripotent stem cells, and reagents used in generating therapeutic cells in vitro, such as IL-4, IFNγ, BMP4, bFGF, VEGF, IL-6, IGF1, IL-11, SCF, or EPO. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Differentiation of Human ES Cells into Transplantable Plasma Cells that Produce Broadly Neutralizing Antibodies

The following example describes the differentiation of human ES cells into transplantable plasma cells that produce broadly neutralizing antibodies.

Bone Marrow Long-Lived Plasma Cells Maintain Serum Antibodies after Infection or Vaccination.

Most clinically-used vaccines depend upon the production and maintenance of neutralizing antibodies. As a result, the major correlates of protection following vaccination are the quantity, affinity, epitope-specificity, and duration of plasma cell-derived antibodies elicited by the immunization. Plasma cells are terminally differentiated B lymphocytes that secrete several thousand antibody molecules per second and devote nearly 70% of their transcriptome to antibody synthesis. During the first few days of a T cell-dependent antibody response, plasma cells survive for only several days before undergoing apoptosis. Other plasma cells that express progressively higher affinity antibodies are generated later from the germinal center reaction. These affinity-matured plasma cells can persist up to decades, and reside mostly in the bone marrow. These long-lived plasma cells constitutively secrete antibodies irrespective of the presence of antigen and without cellular division and are the major determinants of humoral immunity. Because of their prolific rate of antibody secretion, it was shown that just 1 μl of immune serum, corresponding to antibodies derived from only ˜3 antigen-specific long-lived plasma cells, is sufficient to protect naive mice from an otherwise lethal West Nile virus infection. The data as described herein demonstrates the protective potency of these cells if they express neutralizing antibodies.

Methods to Generate Definitive Hematopoietic Precursors from hES Cells.

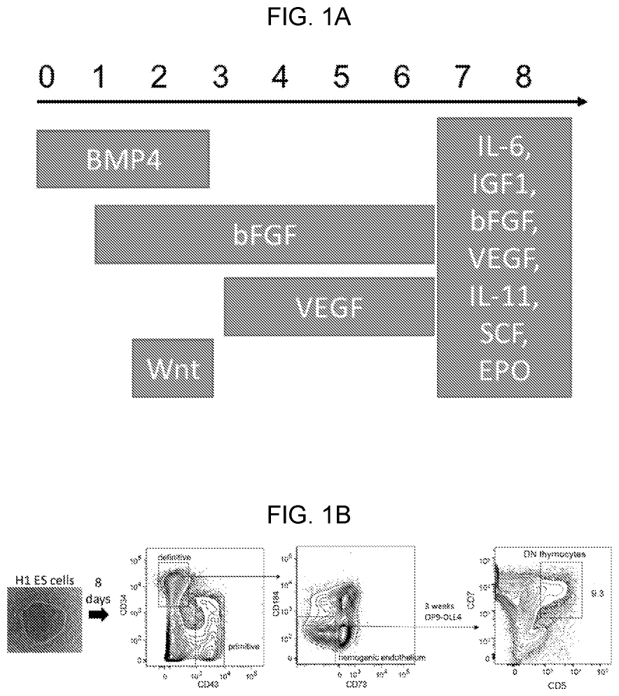

To develop a scalable source of antigen-specific transplantable long-lived plasma cells, H1 human embryonic stem cells were utilized. These are the best characterized human ES cells, have minimal lineage bias, and readily generate hematopoietic cells. Hematopoiesis occurs in at least two separate waves: primitive and definitive. Primitive hematopoiesis generates a restricted set of erythroid and myeloid lineages. In contrast, definitive hematopoiesis ultimately gives rise to adult-type blood cells, including lymphocytes. Thus, a major therapeutic goal for hES cell work has been to generate definitive hematopoietic precursors. Methods to reproducibly generate definitive hemogenic endothelium, a precursor to definitive hematopoietic cells, using H1 hES cells have been optimized as described by Sturgeon et al, Nat Biotech, 2014. The method employs sequential manipulation of signals during mesoderm induction, including the crucial step of canonical WNT signaling activation at Day 2 of the process (see e.g., FIG. 1A). This eventually allows for the generation of T cells, even by pluripotent lines that otherwise lack this potential (see e.g., FIG. 1B). Importantly, these differentiations are performed under defined serum-free conditions, thereby maximizing reproducibility and allowing for facile troubleshooting.

Development of Approaches to Yield B Cells from Definitive Hematopoietic Precursors.

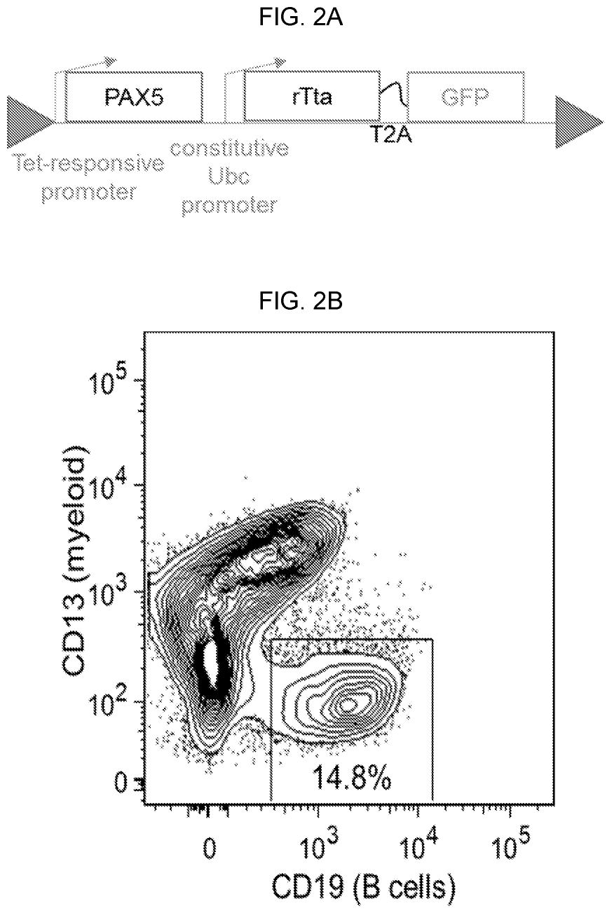

Having generated definitive hematopoietic progenitors, methods to differentiate these precursors into B cells were explored. Very few studies to date have reported robust B cell differentiation from human ES cells, and these approaches either failed to generate mature B cells or only could be performed on one specific iPS line. None of these approaches used defined conditions, and adapting these approaches to any of the presently tested hES cell lines were unsuccessful. Therefore, a combination of hemogenic endothelium were generated (see e.g., FIG. 1), followed by expression of PAX5, a B lineage-promoting transcription factor. Definitive hemogenic endothelial cells were transduced with lentiviral vectors encoding constitutively expressed rTTA-T2A-GFP, a doxycycline-inducible transcriptional activator linked to a reporter by a ribosome skipping 2A sequence, and PAX5 driven by a doxycycline-inducible promoter (see e.g., FIG. 2A). Transduced cells thus constitutively express rTTA and GFP, but express PAX5 only upon doxycycline treatment. After co-culture with Flt3L, SCF, IL7, and MS5 stromal cells for 20 days, B cells were only observed upon doxycycline treatment (see e.g., FIG. 2B). Thus, defined transcription factor expression can promote B lymphopoiesis by otherwise recalcitrant progenitors.

CRISPR/Cas9-Mediated Introduction of Influenza-Specific Antibody Genes into Endogenous Loci of hES Cells.

Previous attempts to generate B cells from hES cells have yielded immature lymphocytes with no surface antigen receptor expression. To address this problem and to provide pathogen-specificities to developing B cells, targeting cassettes containing VDJ and VJ sequences from F16, a broadly neutralizing influenza-specific antibody, and zeocin and hygromycin resistance cassettes, respectively were generated (see e.g., FIG. 3A). These targeting cassettes were nucleofected into H1 hES cells along with Cas9 and guide RNAs (gRNAs) to target the endogenous immunoglobulin heavy chain and kappa light chain loci (ZFN or TALENs can also be used). Cells were selected for zeocin and blasticidin resistance, and PCR analyses were performed to confirm proper targeting (see e.g., FIG. 3B). These cells were then transfected with Cre recombinase-encoding constructs, and subclones lacking the drug resistance cassette were isolated (see e.g., FIG. 3C showing PCR confirmation of IgH and Igk targeting with F16 genes and removal of drug resistance cassette).

Methods to Generate Long-Lived Plasma Cells from B Cells.

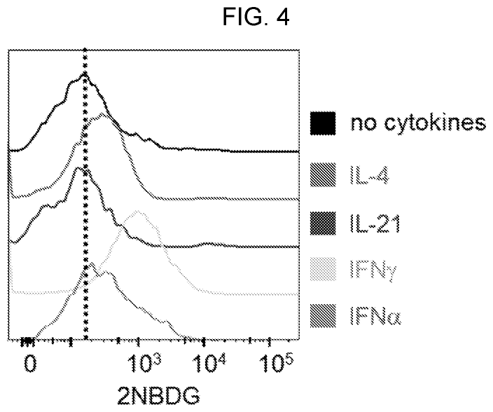

Cues that promote long-lived, rather than short-lived plasma cell formation from B cells in vivo were recently identified. It was shown that long-lived plasma cells import far more glucose than do their short-lived counterparts. This enhanced glucose uptake promotes elevated antibody secretion and mitochondrial pyruvate for respiration, and is required for long-term plasma cell persistence. It is currently believed enhanced glucose uptake is the only known property that allows for prospective separation of short- from long-lived plasma cells. Through an in vitro screen of extrinsic factors, it was discovered that IFNγ and IL-4 promote glucose uptake (see e.g., FIG. 4).

Xenotransplantation Models to Assess Plasma Cell Longevity.

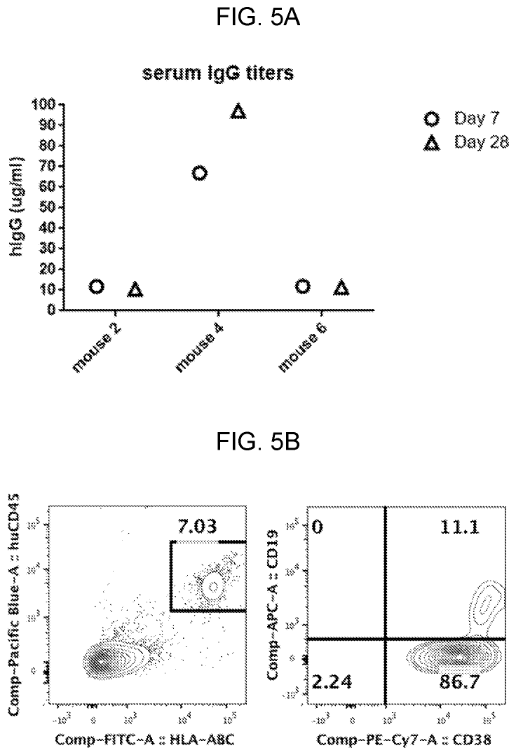

Several protocols, in addition to the work described above, have reported methods to allow for prolonged survival of human plasma cells in vitro. Yet the lack of a xenotransplantation model for human plasma cells has precluded analysis of survival in vivo. To address this problem, a system in which adherent fibroblast/mesenchymal stem cells from human bone marrow are transplanted subcutaneously into immunodeficient NOD-SCID IL2rγ^{-/-} (NSG) mice was developed. Bone-like ossicles form ˜8 weeks after transplantation, providing a fully human microenvironment. These ossicles robustly support normal human hematopoiesis as well as primary myeloid malignancies, some of which had never previously grown in xenotransplants. To test if these ossicles also support normal plasma cell survival, 1-5×10⁶ primary human bone marrow CD138⁺ plasma cells were injected directly into ossicles. Serum human IgG was observed at 2 and 4 weeks after transplantation, with no decline in antibody concentrations between these timepoints (see e.g., FIG. 5A). At 4 weeks, ossicles were harvested for flow cytometric (see e.g., FIG. 5B) and ELISPOT (data not shown) analysis of plasma cells. Antibody-secreting cells were observed in all injected ossicles. These data demonstrate that human ossicles support normal long-lived plasma cell survival and function. It is believed that this is the first such system of its kind.

Generate Pathogen-Specific B Cells from hES Cells.

Defined transcription factor expression is used to drive B cell development from F16 knockin hES cells as in FIG. 2-FIG. 3. The use of genomically integrated lentiviruses may provide for a problematic translational strategy. A such, non-integrating approaches, such as modified RNAs, to transiently introduce factors only when they are needed for differentiation and lineage commitment can be used. In support of this, PAX5 expression mediates a positive feedback loop with EBF1 to maintain B lineage commitment. Thus, transient expression of exogenous PAX5 may trigger stable maintenance of endogenous PAX5 expression. To define the window during which exogenous PAX5 expression promotes B cell commitment, doxycycline will be administered only from D1-7, D8-14, or D15-21 of co-culture with MS5 cells. Similar experiments will be performed with EBF1-encoding lentiviruses. CD19⁺ B cells will be identified by flow cytometry, and expression of influenza-specific IgM will be confirmed using a hemagglutinin tetramer reagent or using methods and reagents known in the art.

Though Pax5 expression promotes B lineage commitment in the present system, if preferential development of B-1a cells could occur. These CD5-expressing B-1a cells are derived from early fetal precursors and not from adult stem cells. B-1a cells have not been shown to possess the ability to generate long-lived plasma cells. If predominantly B-1a cell differentiation is observed in the PAX5 cultures, further differentiation of hemogenic endothelium cells towards definitive hematopoiesis using Notch signals can be performed. As shown above, hemogenic endothelium gives rise to T cells when co-cultured with OP9 cells expressing the Notch ligand Delta-like 4 (DL4). Notch ligands promote T cell development in two stages. First, Notch signals are essential to promote definitive hematopoiesis. Second, Notch signals promote T cell commitment from hematopoietic progenitors. To define the window of Notch signaling that is required for definitive hematopoiesis, hemogenic endothelium will be transduced with doxycycline-inducible PAX5 lentiviral vectors, and then co-cultured on OP9-DL4 cells for 1-10 days. This transient period of Notch signaling has been shown to promote HSC-like cells, from which conventional B-2 and B-1 b cells are derived. Transduced cells will be purified by FACS and transferred to MS5 cells for B cell differentiation for 20 days in the presence of doxycycline. B cells will be checked for CD5 expression to quantify B-1a frequencies.

Generation of Long-Lived Plasma Cells from Mature Human B Cells.

In addition to generating mature B cells as described above, primary tonsillar naïve B cells are used to optimize differentiations to long-lived plasma cells using the methods of the present invention. CD20⁺ CD27⁻ B cells are sorted onto NIH 3T3 cells expressing human CD40L and BAFF. Either IL-4 or IFNγ are added to the cultures at concentrations from 1-100 ng/ml. At 6 days, CD38^{high}CD27^{high} plasmablasts/plasma cells are injected intravenously or directly into ossicles in NSG mice as in FIG. 5. Serum human IgG is sampled biweekly. If serum antibodies are maintained over the course of 8-16 weeks, animals are sacrificed and ossicles are harvested for flow cytometric and ELISPOT analysis as in FIG. 5.

There is a possibility that the in vitro-derived plasma cells may not persist upon transplantation in vivo, despite enhanced glucose uptake. In parallel to the in vitro culture approaches outlined above, culture methods reported by others will be employed. Transwell culture methods are available in which plasma cells can be maintained for months in vitro. In this approach, fibroblastic L cells are engineered to express CD40L, and cells are cultured in the presence of IL-21. At later timepoints, plasma cells are separated from stromal cells via a transwell filter. Through periodic media changes, these cells achieve quiescence and can be maintained for months. Using this differentiation system, plasma cells will be transplanted into ossicles for long-term maintenance in vivo.

Treatment of Infection (e.g., Influenza).

The hES cells are differentiated into long-lived plasma cells, and transplanted into NOD-SCID-IL2rg (NSG) mice carrying a human ossicle in order to validate this method in an animal model. These ossicles are formed after transplanting mice with mesenchymal cells from human marrow (as described above). After one or more weeks (enough time to allow flu-specific antibody concentrations to rise) these mice are infected with a dose of flu that would normally be lethal for NSG mice. Survival and flu virus burden in the mice are then measured.

For therapeutic use in humans, hES cells modified into long-lived plasma cells, able to avoid immune rejection, are administered prophylactically as a vaccine.

Chronic Infection Treatment.

Young NSG mice are reconstituted simultaneously with cord blood CD34+ cells (intravenously) and with mesenchymal cells (as described above) to form ossicles. After T cell reconstitution, mice will then be infected with HIV to hit the human CD4+ T cells, and then broadly neutralizing HIV-specific plasma cells will be transplanted. HIV titers and antibodies will be measured to see how well ongoing infections are suppressed. A mouse model as described in Halper-Stromberg et al. 2014 Cell 158(5)989-999 can be utilized. Once the universal cells are developed, this is tested in non-human primates and SIV.

For therapeutic use, the cells of the present invention are administered to a patient having chronic infection, regardless of whether the virus is active or dormant.

Enzyme Replacement Therapy.

A mouse model of Hurler's syndrome (Clark et al. 1997 Science 6(4) 503-11), in which there is a deficiency of α-L-iduronidase is used. Systemic enzyme replacement is therapeutic for Hurler's syndrome. The mouse model is crossed to NSG, ossicles formed (as described above), and plasma cells engineered to secrete α-L-iduronidase instead of antibodies (through genetic replacement or IRES knockins downstream of Aβ genes) are transplanted. Enzyme concentrations and brain neuropathology (e.g., lysosomal distension) are measured.

Treatment of Cancer.

NSG/ossicle mice (as described above) are engrafted with primary Non-Hodgkins Lymphoma, and then administered plasma cells expressing rituxan (Chao et al. 2010 Cell 142(5) 699-713). Tumor burden is quantified histologically and by flow cytometry. If a suicide gene is built in, the plasma cells can be removed whenever residual disease is gone.

Treatment of Autoimmune Disease.

NSG/ossicle mice are transplanted with plasma cells that express Soliris/eculizimab. These mice are treated with anti-Gq1b antibodies and measure motor function and paralysis. This mouse model of autoimmune disease described in Halstead et al. 2008 131(Pt5)1197-208).

Treatment Using Therapeutic Antibodies.

The cells and methods of the present disclosure can be used in treatment of a subject in need of a therapeutic antibody. As an example, a therapeutic antibody can be overexpressed on the modified ES cell as described herein. For example, treatment of Alzheimer's disease can be accomplished by engineering the modified ES cells of the present invention to express a therapeutic antibody, such as aducanumab to avoid the buildup of Aβ plaques.

Materials and Methods.

These studies as described herein use H1 (WA01, NIH registration #0043) cells for modifications and differentiations, a male-derived line. However, all mouse experiments utilize both males and females, and are included as a variable in the experiments. Moreover, donor xenografts are derived from both males and females. Y-chromosome PCR is performed to determine the sex of the anonymous donor, and again these data are included as a variable in the analysis.

Description of procedures: The majority of the work is performed in vitro for hES cell differentiations and genetic modifications. Modest numbers of immunocompromised NOD-SCID/IL2rγc-deficient (NSG) mice will be used as recipients for ossicles and plasma cells, and for teratomas. A smaller number of wild-type 057B16 mice will be used for teratoma recipients. Recipients will be 8 weeks-old and of both sexes.

These studies in mice are a prelude to the clinical development of cell-based vaccines. Studies in mice have proved highly relevant to such applications in the past and a great deal is known about the cellular, molecular, and genetic aspects of mouse immunity. Studies in mice can be done efficiently at relatively low cost, given the potential benefits.

Procedures to alleviate pain or discomfort: Every effort is made to ensure that the level of pain and discomfort in mice is minimal. Mice are anesthetized with inhaled isofluorane prior to retroorbital injections. Animals with teratomas of 20 mm diameter will be sacrificed. III appearing mice are sacrificed, and sentinel cages are used routinely in the animal facility.

Animals are euthanized by inhalation of CO in accordance with the recommendations of the Panel on Euthanasia of the American Veterinary Medical Association.

### Example 2: Genetic Modification of Human ES Cells to Reduce Immunogenicity

The following example describes the genetic modification of human ES cells to reduce immunogenicity.

Universal-donor hES cells are a goal of many fields, but the immunological barriers are substantial. Human pluripotent stem cells grow indefinitely, thus addressing one aspect of scalability for plasma cell therapies. However, unless the stem cell line is autologous, immune suppression will necessarily be required to prevent graft rejection. This would almost certainly limit such therapies to immediately life-threatening disorders. Autologous iPS therapies, while perhaps not subject to immune rejection, are not scalable to the general population. To address these issues, the creation of ‘universal’ donor cell lines is proposed herein. These lines are genetically stripped of their immunogenicity, thereby allowing cells and tissues derived from a single line to be transplanted into any recipient. Removal of HLA expression has been shown to be a requirement for such a universal donor line, but mouse allograft studies have demonstrated that this alone is insufficient to prevent rejection. Subsequent research has implicated antibodies, complement, NK cells, and phagocytes as other mediators of graft rejection. Beginning with an HLA-deficient line, the reactivity of hES cells to each of these other immune mediators of rejection have been identified.

Materials and methods are as described above unless noted otherwise.

Generation of an HLA-Deficient hESC Line.

A robust workflow was optimized to generate targeted mutations in human ES cells. A Cas9-expression construct and up to 3 different gRNA-encoding vectors were co-transfected into H1 human ES cells. Individual colonies were manually picked and subjected to Miseq analysis of targeted genes to identify clones carrying frameshift mutations introduced by non-homologous end-joining errors. Candidate clones were then plated at exactly 1 cell/well by fluorescence activated cell sorting (FACS), and Miseq analysis was again performed to confirm the mutations and lack of mosaicism (see e.g., FIG. 6A). Next, clones were expanded, karyotyped, and their differentiation potential towards hematopoietic lineages was confirmed. Through CRISPR/Cas9-based targeted mutations, a karyotypically normal hES cell line that lacks HLA expression was generated. One clone carrying inactivating mutations in both alleles of β2m and TAP1 and one allele of CD74 was identified through MiSeq analysis of the targeted regions (see e.g., FIG. 6B). In this clone, Interferon-γ (IFNγ)-induced HLA-I expression was completely abrogated (see e.g., FIG. 6C), and a normal karyotype was confirmed. Monocytes derived from this HLA-I-deficient line failed to stimulate allogeneic primary CD8⁺ T cell proliferation (see e.g., FIG. 6D showing HLA-deficient hES cell progeny do not stimulate T cells). This HLA-I-deficient line was subsequently re-targeted using CRISPR to ablate the remaining allele of CD74 and both alleles of CIITA, a transcription factor required for expression of HLA-II. This clone was also confirmed to possess a normal karyotype. Subsequent analysis confirmed that after each genetic modification, cells retained hematopoietic potential, and derivative monocytes lacked detectable expression of HLA-II (see e.g., FIG. 6E showing HLA-II is not expressed by CIITA/CD74-deficient cells).

Generation of AAVS-Targeting Immune Evasion Cassettes.

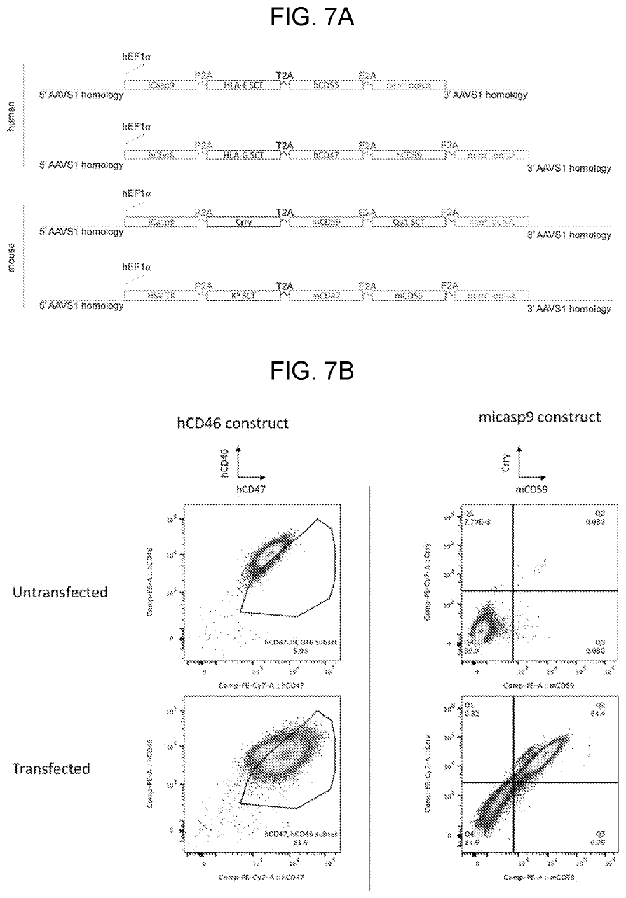

It is believed that ablation of HLA-I expression renders cells susceptible to NK cell lysis. Ligands of the activating receptor NKG2D were ablated (see below). However, NKG2D blockade eliminates only ˜50% of NK cell-mediated specific lysis. Thus, additional steps will be taken to further eliminate NK cell reactivity, for example, through the expression of HLA-E and HLA-G covalently-linked to peptide and β2m to form single chain trimers. These trimers do not exchange peptide or β2m with endogenous HLA, and inhibit NKG2A- and ILT2-expressing NK cells. Other aspects of the immune response, including antibody binding, complement deposition and macrophage phagocytosis can also contribute to rejection. To address and evade each of these immune mechanisms, a set of multicistronic vectors were constructed for targeting the human AAVS locus (see e.g., FIG. 7A, TABLE 1). This locus is considered as a ‘safe harbor’ that is protected from silencing. Flow cytometric analysis confirmed expression in HLA-deficient hES cells see e.g., FIG. 7B). A summary of immune evasion and suicide genes and targets can be found in TABLE 1.

TABLE 1
Human and mouse genes to be expressed in AAVS locus.

| Gene name | Pathway/Cell Type Inhibited |
|---|---|
| HLA-E single chain trimer (Qal in mouse) | NKG2A+ NK cells |
| HLA-G single chain trimer (human only) | ILT2/KIR2DL4+ NK cells |
| K-b single chain trimer (mouse only) | Ly49C+ NK cells |
| CD46/Crry (in mouse) | complement/C3b and C4b |
| CD55 | complement/C3 convertase |
| CD59 | complement/C9 |
| CD47 | phagocytosis |

Sucide genes to be delivered to AAVS locus

| Gene name | Death-inducing drug |
|---|---|
| iCasp9 | AP1903 |
| HSV thymidine kinase | ganciclovir |

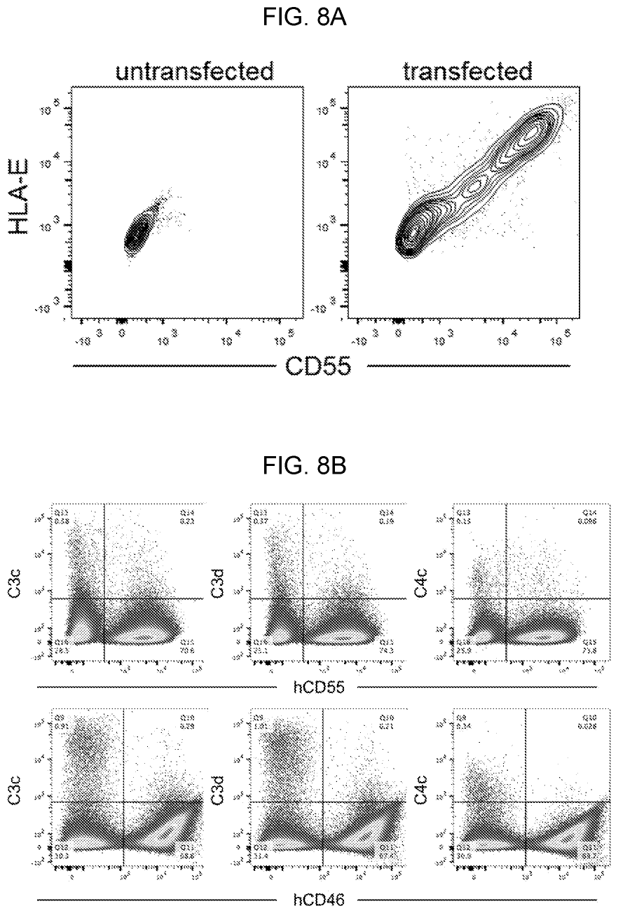

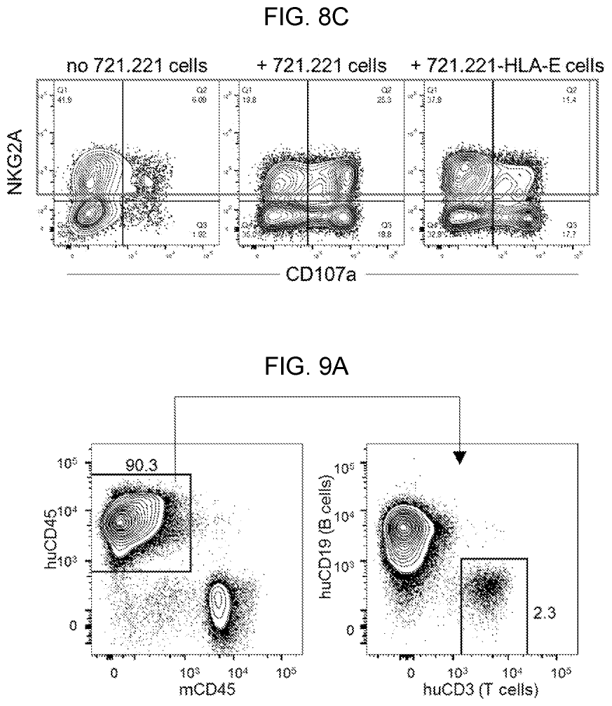

The targeting constructs were validated through transfection into CHO cells and assessing the expression of immune evasion genes by flow cytometry. The results showed that human CD55 and HLA-E single chain trimer displayed tight co-expression (see e.g., FIG. 8A), confirming the function of the 2A sequences and construct 1 (SEQ ID NO: 1) in FIG. 7. To confirm the correct function of the inserted gene products, a stable hCD55-expressing CHO line through neomycin selection was identified. These cells were then stained with anti-CHO antibody, incubated with human C7-deficient serum, and finally stained for C3c, C3d, and C4c deposition. Expression of hCD55 and hCD46 abrogated complement deposition, whereas the residual hCD55-cells in the culture displayed robust complement deposition (see e.g., FIG. 8B). These data confirm the functionality of the hCD55 transgene. To confirm that the HLA-E single chain trimer functions correctly, stable cell lines in 721.221 cells, a NK cell-susceptible target cell line, were generated. Incubation of unmodified 721.221 cells with primary PBMCs led to robust NK cell degranulation, as measured by cell surface CD107a staining. However, co-incubation with HLA-E-expressing 721.221 cells specifically inhibited NKG2A+NK cell degranulation (see e.g., FIG. 8C).

Generation of NKG2D Ligand-Deficient hES Cells.

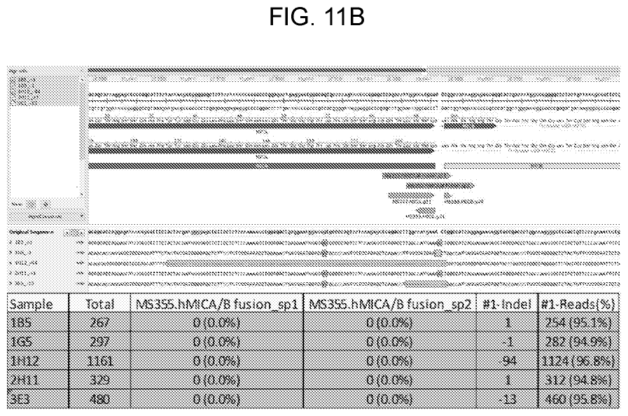

As the absence of HLA-I may render target cells susceptible to NK cell-mediated cytolysis, NKG2D ligands are targeted through CRISPR/Cas9. Of these, RNA-seq analysis demonstrates that only MICA and MICB are expressed in long-lived plasma cells. Preliminary sequencing of ˜400 nucleofected clones revealed one line carrying frameshift mutations in 2 alleles of MICA and MICB, and a large deletion in the other chromosome, creating an in-frame fusion between MICA and MICB (FIG. 11A). This MICA/B fusion was retargeted with CRISPR/Cas9, to generate a clone with frameshift mutations in all 4 alleles of MICA and MICB (FIG. 11B). This clone was validated for normal karyotype. HLA, MICA/B deficient hES cells will henceforth be referred to as HM-KO hES.

Generation of Stable AAVS Immune Evasion Transfectants in HLA/MICA+MICB KO (HM-KO) hES Cells.

Starting with HM-KO hES cells, two sets of nucleofections were performed. One nucleofection was performed using the human immune evasion cassettes shown in FIG. 7A, while a separate nucleofection was performed using the mouse immune evasion cassettes (mAAVS). These constructs were co-transfected with a Cas9 expression construct and gRNAs targeting the AAVS locus. Cells were selected for neomycin and puromycin resistance, and expression of genes was confirmed by flow cytometry (see e.g., FIG. 7B).

Test Teratoma Formation In Vivo.

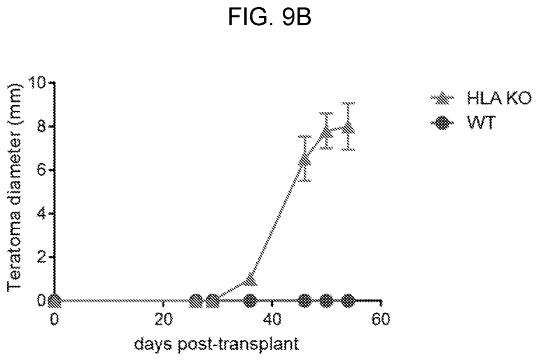

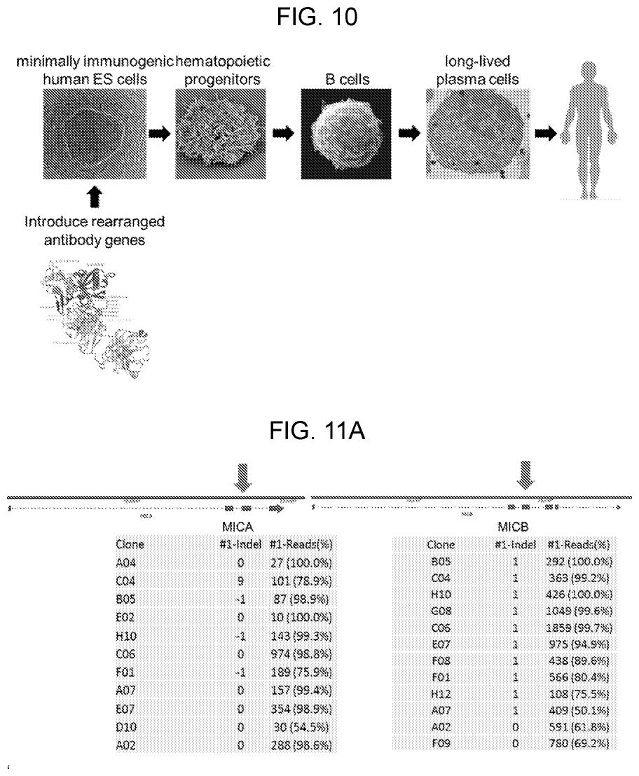

To test human immune evasion in vivo, humanized NSG W41 mice were generated. Approximately 2×10⁵ cord blood CD34+ cells (obtained from the Saint Louis Cord Blood Bank) were transplanted into unconditioned NSG W41 mice (see e.g., FIG. 9A). Mice were bled to confirm human reconstitution, and 1 million cells of each version of the modified hESCs were embedded in Matrigel and transplanted subcutaneously into humanized NSG-W41 mice. Teratoma growth was monitored over the course of 12 weeks, or until tumors reached a mass of 20 mm, at which point mice were euthanized. Humanized mice inoculated with HLA-deficient cells, but not unmodified hES cells, formed teratomas (see e.g., FIG. 9B showing HLA-KO hES cells evade rejection by humanized mice). In certain experiments, mice will be treated with AP1903 to activate iCasp9 and initiate teratoma regression. Some aspects of the immune response are not functional in humanized NSG W41 mice. For example, NK cells form poorly in this system, and antibody responses are minimal. Therefore, teratoma assays in C57Bl6 mice using HM-KO cells stably expressing mAAVS constructs will be performed. Rejection is expected to be delayed in this xenogenic setting.

It is expected that the modified hES cells evade immune recognition and thus form teratomas in humanized NSG-W41 mice.

1. A method of differentiating human embryonic stem cells (ES cells) into a plasma cell comprising:
generating a progenitor cell; and
differentiating the progenitor cell into a B cell.

2. The method of claim 1, wherein
the progenitor cell is a hemogenic progenitor cell or a hemogenic endothelial cell; or
the plasma cell produces a therapeutic agent selected from a protein, a broadly neutralizing antibody, or an enzyme.

3. The method of claim 1, wherein differentiating the progenitor into a B cell comprises:
expressing a B lineage-promoting genetic factor driven by an inducible promoter; and
activating the progenitor with a stimulus, resulting in B cell differentiation.

4. The method of claim 3, wherein the genetic factor is selected from one or more of the group consisting of PAX5, EBF1, FOXO1A, BCL11A, TCF3, IKZF1, IRF4, IRF8, and SPI1; or the stimulus is selected from doxycycline or tetracycline.

5. The method of claim 3, comprising co-culturing the progenitor cell with (i) a cytokine selected from one or more of the group consisting of: Flt3L, SCF, and IL-7; and (ii) MS5 stromal cells, for a period of time sufficient to promote B lymphopoiesis.

6. The method of claim 1, wherein the human ES cell is nucleofected with a pathogen-specific antibody gene-encoding cassette.

7. The method of claim 6, wherein the pathogen specific antibody cassette comprises VDJ and VJ sequences selected from one or more of the group consisting of flu antibody, HIV antibody, or flavivirus antibody.

8. The method of claim 7, wherein the pathogen specific antibody cassette comprising VDJ and VJ sequences are selected from one or more of the group consisting of F16, VRC07, 10E8, N6, 3BNC117, EDE1, and C10.

9. A method of generating a long-lived plasma cell comprising:
(i) administering IFNγ or IL-4 to B cells derived from human ES cells; or
(ii) administering IL-21 to 3T3 fibroblast cells engineered to express CD40L and BAFF.

10. The method of claim 9, comprising: providing primary tonsillar naïve B cells; and introducing IL-4 or IFNγ to the primary tonsillar naïve B cells.

11. The method of claim 9, wherein the long-lived plasma cell has increased antibody, protein, or enzyme secretion; increased mitochondrial pyruvate for respiration; or increased glucose uptake relative to a wild-type plasma cell.

12. A method of treating a subject in need thereof comprising administering a therapeutically effective amount of a plasma cell to a subject,
wherein,
the subject has a virus and the plasma cell expresses sequences from an antibody broadly neutralizing the virus;
the subject has an enzyme deficiency and the plasma cell secretes an enzyme; or
the subject has an autoimmune disease, neurodegenerative disorder, or cancer and the plasma cell expresses an immunotherapeutic agent, optionally selected from aducanumab, rituxan, or eculizimab.

13. (canceled)

14. A genetically engineered stem cell comprising:
(i) reduced expression of one or more HLA-I and HLA-II relative to a wild-type stem cell; or
(ii) constructs encoding genes leading to evasion of complement fixation, evasion of NK cell recognition, or evasion of phagocytosis.

15. The genetically engineered stem cell of claim 14, wherein
(i) the genetically engineered stem cell has an increased expression of one or more immune evasion factors relative to a wild-type stem cell;
(ii) the genetically engineered stem cell has an increased expression of one or more of HLA-E and HLA-G relative to a wild-type stem cell;
(iii) the immune evasion factors are selected from one or more of the group consisting of HLA-E, HLA-G, CD46/Crry, CD47, and CD55;
(iv) the immune evasion factors are selected from one or more of the group consisting of CD46/Crry and CD55;
(v) the immune evasion factors inhibit immune rejection or promote immune evasion; or
(vi) the stem cell is a human stem cell, an embryonic stem cell, a pluripotent stem cell, or is hypoimmunogenic.

16-19. (canceled)

20. The genetically engineered stem cell of claim 14, comprising an inactivating mutation in encoding genes selected from one or more of the group consisting of: β2 microglobulin, TAP1, CD74, CIITA, and a ligand of NKG2D.

21. The genetically engineered stem cell of claim 20, wherein
(i) the inactivating mutation is in encoding genes TAP1 or CD74;
(ii) the ligand of NKG2D is optionally selected from one or more of the group consisting of MICA, MICB, Raet1e, Raet1g, Raet11, Ulbp1, Ulbp2, and Ulbp3;
(iii) β2 microglobulin and TAP1-encoding genes are genetically modified to eliminate HLA-I expression and prevent direct recognition by allogeneic CD8+ T cells;
(iv) CD74-encoding genes are genetically modified to eliminate HLA-II expression; or
(v) NKG2D ligand encoding genes are genetically modified to evade natural killer cell recognition;
wherein, the genetically engineered stem cell produces a progeny and the progeny exhibits reduced immunogenicity.

22-23. (canceled)

24. The genetically engineered stem cell of claim 14, comprising
(i) at least one immune evasion gene selected from the group consisting of: HLA-E single chain trimer, HLA-G single chain trimer, K-b single chain trimer, CD46/Crry, CD55, and CD47; or
(ii) at least one immune evasion gene inhibiting a pathway or a cell type selected from the group consisting of NKG2A+NK cells, ILT2/KIR2DL4+NK cells, Ly49C+NK cells, complement/C3b and C4b, complement/C3 convertase, complement/C9, and phagocytosis; and
(iii) optionally, at least one suicide gene.

25. The genetically engineered stem cell of claim 14, wherein the genetically engineered stem cell comprises a drug-inducible suicide gene selected from one or more of the group consisting of: iCasp9, HSV thymidine kinase, cytosine deaminase, and E. coli nitroreductase and the drug is selected from the group consisting of AP1903, ganciclovir, 5-fluorocytosine, and CB1954.

26. A method for making a genetically engineered stem cell comprising:
(i) introducing a genetic modification or an inactivating mutation to reduce immunogenicity; or
(ii) delivering a construct to a safe harbor locus and encoding genes that lead to evasion of complement fixation, NK cell recognition, or phagocytosis, wherein
(i) the immunogenicity of the cell reduced by elimination of HLA-I expression, elimination of HLA-II expression, and elimination of natural killer cell recognition;
(ii) the inactivating mutation is in encoding genes selected from one or more of the group consisting of: β2 microglobulin, TAP1, CD74, CIITA, and a ligand of NKG2D, the ligand of NKG2D is optionally selected from one or more of the group consisting of MICA, MICB, Raet1e, Raet1g, Raet11, Ulbp1, Ulbp2, and Ulbp3;
(iii) the gene leading to evasion of complement fixation is selected from one or more of the group consisting of CD46/Crry, CD55, and CD59;
(iv) the gene leading to evasion of NK cell recognition is selected from one or more of the group consisting of HLA-E and HLA-G; or
(v) the gene leading to evasion of phagocytosis is CD47.

27. (canceled)

28. A method of treating a subject in need thereof with a genetically engineered stem cell or progeny of a genetically engineered stem cell of claim 14 wherein,
(i) the stem cell is transplanted into a subject;
(ii) a tissue of a subject is destroyed by an autoimmune disease;
(iii) a tissue of a subject destroyed by an autoimmune disease is regenerated;
(iv) pancreatic cells or oligodendrocytes are regenerated;
(v) the subject has an autoimmune disease, cancer, a neurodegenerative disease;
(vi) the subject has type I diabetes, multiple sclerosis, a pathogen, or an infectious disease;
(vii) humoral immunity is generated; or
(vii) antibody-mediated immunity is generated; and
optionally, wherein host immunosuppression is absent.

29-30. (canceled)
